(19) United States
(12) Patent Application Publication
(10) Pub. No.: US 2012/0190036 A1
(43) Pub. Date: Jul. 26, 2012

(54) CLINICAL METHOD FOR GENOTYPING LARGE GENES FOR MUTATIONS THAT POTENTIALLY CAUSE DISEASE

(76) Inventors: Poh San Lai, Singapore (SG); Onn Siong Yim, Singapore (SG); Eric Yap, Singapore (SG)

(21) Appl. No.: 13/499,214

(22) PCT Filed: Sep. 29, 2010
(86) PCT No.: PCT/SG2010/000370

§ 371 Date: Mar. 29, 2012

(30) Foreign Application Priority Data
Sep. 29, 2009 (SG) 200906525-1

(51) Int. Cl. C12Q 1/68

(52) U.S. Cl. 435/6.12

(57) ABSTRACT

A method of determining polymorphisms within a large gene comprising the steps of: (a) making a Whole-Genome Amplification (WGA) to obtain sufficient amounts of genetic templates for DNA analysis; (b) enriching the WGA sample with nested primers designed for the large gene; (c) using the enriched WGA sample for high resolution melt (HRM); and (d) detecting differential melt profiles during the transition from double strand to single strand with an increase in temperature wherein sequence point mutations within the gene affects the thermal stability and gives a different melt profile from the normal non-mutated gene sequence, and kits to carry out detection of the same. The method may further comprise the step of spiking the DNA being screened using DNA from a phenotypically normal individual in order to induce synthetic heterozygosity. The method in (d) may also work directly on genomic samples without WGA step if sufficient DNA material is present.

### CROSS-REFERENCE TO RELATED APPLICATION

This application claims benefit of, and priority from, Singapore patent application No. 200906525-1, filed on 29 Sep. 2009 the contents of which are hereby incorporated herein by reference.

### FIELD OF THE INVENTION

The present invention relates to screening and prescreening methods for genotyping large genes for mutations that potentially cause disease such as muscle disease and kits for carrying out the same.

### BACKGROUND ART

There are more than 40 primary congenital muscle disorders known and the most common is a genetic disease called Duchenne muscular dystrophy (DMD) followed by Becker muscular dystrophy (BMD). Both are allelic disorders caused by mutations in the same gene, the DMD gene of the X chromosome, with the latter having a milder phenotype and slower disease progression. Together, DMD and BMD occur with an incidence of approximately 1 in 3,500 newborn males, accounting for 80% of all new cases of dystrophy and 56% of all new cases of congenital myopathy of all types. DMD is a severe disease resulting in severe and progressive muscle wasting. The patient becomes wheel-chair bound by about 12 years of age, and usually do not survive beyond the second decade of life due to pulmonary or cardiac failure. BMD patients may be ambulant with milder symptoms and survive longer.

The DMD gene responsible for both disorders is the largest known in the human genome spanning 2.3 Megabases (or 2,300 kB) which is encoded by 79 exons. This gene is localized to the X chromosome, hence, the disease is recessive x-linked and affects only males while females can be silent carriers. The gene codes for a muscle protein called dytsrophin which is absent or semi-functional in affected patients. About 60% of DMD and BMD cases are caused by large deletions or duplications, but the remaining mutations are mostly small and are known as point mutations. They involve single or multiple base changes, insertions, deletions or duplications. To detect these unknown mutations, the sequences in all the coding exonic regions as well as the intronic regions (flanking sequences, branch-points, promoter regions, etc.) have to be interrogated. This is akin to looking for a needle in a hay-stack and every single base in the large gene sequences has to be checked. There are no known mutation hot-spots for point mutations in the DMD gene. All the reported mutations so far have been heterogeneous and can occur anywhere in the gene, both in exonic or intronic regions which affect protein functioning.

Mutation screening is important to help confirm the clinical diagnosis, detect carriers, perform genetic counselling and allow for prenatal diagnosis or diagnosis of pre-implantation of embryos. In addition, mutation screening in patients is also useful as there are now various novel molecular therapeutic treatments which are currently in various phases of clinical trials. These treatment approaches aim to restore the function of the defective dystrophin muscle protein by targeting specific classes of mutations in the DMD gene. Hence, it is important to identify the specific underlying mutation in each patient in order to select the best possible treatment approaches in future.

The most widely offered test in diagnostic labs for molecular diagnosis in DMD involves detection of common large deletion/duplication mutations costing about a few hundred US dollars or British sterling pounds. Sequencing analysis is offered only at a few university labs and takes a few months for turn-around of results. Sequencing is expensive and depends on the size of the gene and number of exons to be analysed, costs escalating with larger sequences. As the DMD gene is very large with a large number of exons sequencing of the gene costs more than 10 times that of testing for common deletion/duplication mutations. Hence, to currently test for uncommon point mutations it is expensive, time consuming and limited to very few locations.

In mutation screening or genotyping, large genes are challenging to interrogate when a large region has to be analysed quickly in a short time. For typical diagnostic labs offering genetic service, results for patients have to be reported as soon as possible. Prenatal diagnosis and embryo pre-implantation diagnosis require urgent testing within a limited time-frame. While direct DNA sequencing is one of the most common and accurate methods for detecting unknown mutations, this process involves designing individual assays based on location and primer sequences, and subsequently iteratively optimizing each individual assay, and analysing the individual sequences of each region covered by the assays. Thus, it is laborious, costly and time-consuming when a large number of assays and large regions of sequences have to be analysed. Consequently, a large number of mutation screening methods have been introduced to reduce the number of such analyses required by narrowing the region which needs to be sequenced or by using an approach which can easily detect a variant from wild-type sequence. These include denaturing high performance liquid chromatography (DHPLC), single strand conformational polymorphism (SSCP), single condition amplification/internal primer (SACIP), etc. which employs multiple protocol steps and procedures. However, these current methods are not sensitive or efficient enough or suited for high throughput analysis of genes while other chip-based technology are currently too expensive to be affordable for routine diagnosis of heritable genetic disorders in clinics.

Generally detection of mutations in diseases may be required from biological samples from infants or young children. These samples include blood samples, fetuses or embryos. Only very small amount of samples are able to be collected from such sources. All the current detection methods require a significant volume of DNA sample.

High resolution melting (HRM) is a simple, rapid method for mutation scanning which requires template DNA of good quality and quantity. The method involves the melting of double stranded DNA into single stranded DNA with increasing temperature such that amplicons with sequence differences will generate different melt profiles due to the difference in thermal stability. These melt profiles are detected due to the use of saturating intercalating DNA dyes which are released as the DNA strands melt and are captured by new generation melt instruments which are capable of much higher data density capture than conventional real-time PCR machines. The lowest volume sample that can be used to conduct HRM is 10 ng. For small genes or when genotyping a few assays, the DNA amount required is small as compared to assays for DMD gene due to the large size of the gene and the number of assays required to interrogate the entire gene.

### SUMMARY OF THE INVENTION

The present invention seeks to provide novel methods to ameliorate some of the difficulties with the current detection and pre-screening of disease caused by a genetic mutation in a large gene sequence.

We have developed a method that can enrich the DNA such that only a small volume is required that may accurately and quickly detect mutations within a large gene sequence.

Accordingly the present invention provides a method of determining sequence variants within a large gene comprising the steps of (a) enriching a nucleic acid sample of the large gene with nested primers designed for the large gene; (b) using the enriched nucleic acid sample for high resolution melt (HRM); and (c) detecting differential melt profiles during the transition from double strand to single strand with an increase in temperature wherein sequence point mutations within the gene affects the thermal stability and gives a different melt profile from the normal non-mutated gene sequence.

A further aspect of the present invention provides a method of determining polymorphisms within a large gene comprising the steps of: (a) making a Whole-Genome Amplification (WGA) to obtain sufficient amounts of genetic templates for DNA analysis; (b) enriching the WGA sample with nested primers designed for the large gene; (c) using the enriched WGA sample for high resolution melt (HRM); and (d) detecting differential melt profiles during the transition from double strand to single strand with an increase in temperature wherein sequence point mutations within the gene affects the thermal stability and gives a different melt profile from the normal non-mutated gene sequence. The method in parts (c) and (d) may also work directly on genomic samples without WGA step if sufficient DNA material is present.

The method may further comprise the step of spiking the sample DNA being screened with DNA from a phenotypically normal individual in order to induce synthetic heterozygosity.

Preferably the large gene is the DMD gene that may potentially contribute to muscle disease or muscular dystrophy such as Duchenne muscular dystrophy or Becker muscular dystrophy.

The method may further comprise a cleaning step prior to the HRM step such as washing with a high magnesium concentration, exo-nuclease digestion, dephosphorylation treatment or filtration.

A further aspect of the present invention provides a kit comprising at least two nested primers specific to a large gene of interest and reagents' for high resolution melt analysis.

A further aspect of the present invention provides a kit comprising reagents, primers and probes for whole genome amplification and high resolution melt analysis, including the nested primers specific to the gene fragment of interest. Preferably the kit is to rapidly detect mutations that may potentially contribute to muscle disease.

The kit may further comprise a DNA isolated from a phenotypically normal individual.

Preferably the at least two nested primers are specific to a DMD gene. Preferably the at least two nested primers specific to the DMD gene are specific to a nucleic acid homologous to a section of SEQ ID. No. 1. preferably selected from any one of the primers listed in table 1 or 3.

Preferably the reagents for whole genome amplification comprise an agent for polymerization.

Preferably the reagents' for high resolution melt analysis comprise intercalating DNA dyes.

The kit may further comprise a washing mix high in magnesium or an exo-nuclease.

kit comprising reagents, primers and probes for whole genome amplification and high resolution melt analysis, including the nested primers specific to the gene fragment of interest. Preferably the kit is to rapidly detect mutations that may potentially contribute to muscle disease.

### BRIEF DESCRIPTION OF THE DRAWINGS

FIG. 1: Depicts a symbolized flow chart of a preferred embodiment of the method of the invention

FIG. 2: Melt-analysis for WGA enriched and purified products 2A depicts results from Exon 48 showing use of normal WGA products (without purification and/or enrichment): the three different genotypes cannot be distinguished from each other. In comparison 2B depicts results Exon 48 showing enriched WGA products: the three different genotypes (A/A, A/C and C/C) cluster in distinct groups. 2C depicts results from Exon 37 showing use of normal WGA products (without purification and/or enrichment): the three different genotypes cannot be distinguished clearly from each other. In comparison 2D depicts results Exon 37 showing enriched WGA products: where the three different genotypes (A/A, A/G and C/C) cluster in distinct group.

FIG. 3. HRM analysis using probes and primers shows use of probes, to differentiate two SNPs (A/G and A/C) at probe melt regions Rs1800274 (A/G) peak A and Rs1800275 (NC) peak B

FIG. 4. HRM analysis using primers shows use of different dyes for high resolution melt to differentiate between normals (N), patient (P) and carrier (C) samples using the DMD primers for Exon 46 where Differentiation of Genotypes are indicated as Mutant (P), Wild Type (N), and Heterozygote carriers (C).

FIG. 5. PCR and HRM were performed using DNA from wild-type/normal individuals samples for validation. FIG. 5A-D HRM results show examples of the tight clustering of melt curves for normal DMD gene amplicons using primers Exon 1P 5A, Exon 4a 5B Exon 19b 5C and Exon 60a 5D. FIG. 5E-H HRM results show examples of the presence of SNPs (variants) which are clearly detectable from the profiles of outliers (in grey and black) in some amplicons in Exon 37c 5E, Exon 48c 5F, Exon 53c 5G, and Exon 79r 5H.

FIG. 6. DNA samples from patients were analyzed and compared against DNA samples from 6 normal individuals (Grey). Red, blue, or green melt curves indicate outliers deviating from the normal samples which act as references. FIG. 6A-H shows the Unspiked HRM results. FIG. 6I-N depicts the Spiked results of samples taken from patients. FIG. 6O & P show samples from known carriers of DMD (family members of the patients) and how they compare to Patient and Wild-Type/Normal samples.

FIG. 7 The results for Patient 414/418/432 show the probe melt peaks FIG. 7A. The mutation creates a mismatch in the patient DNA, causing the probe to melt earlier than the perfect complementary match found in normal samples.

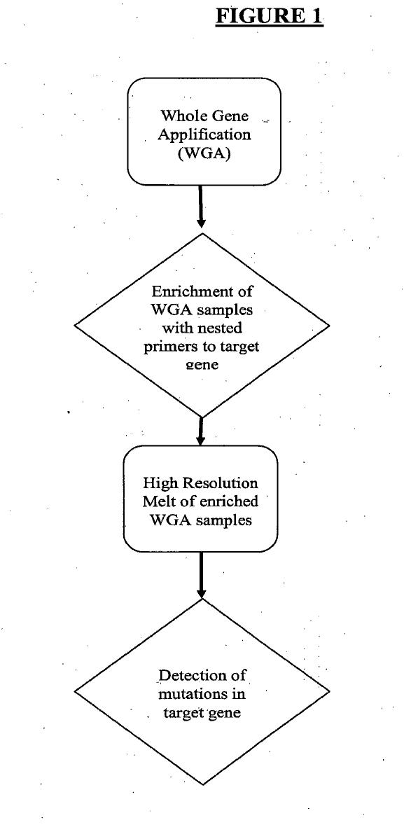

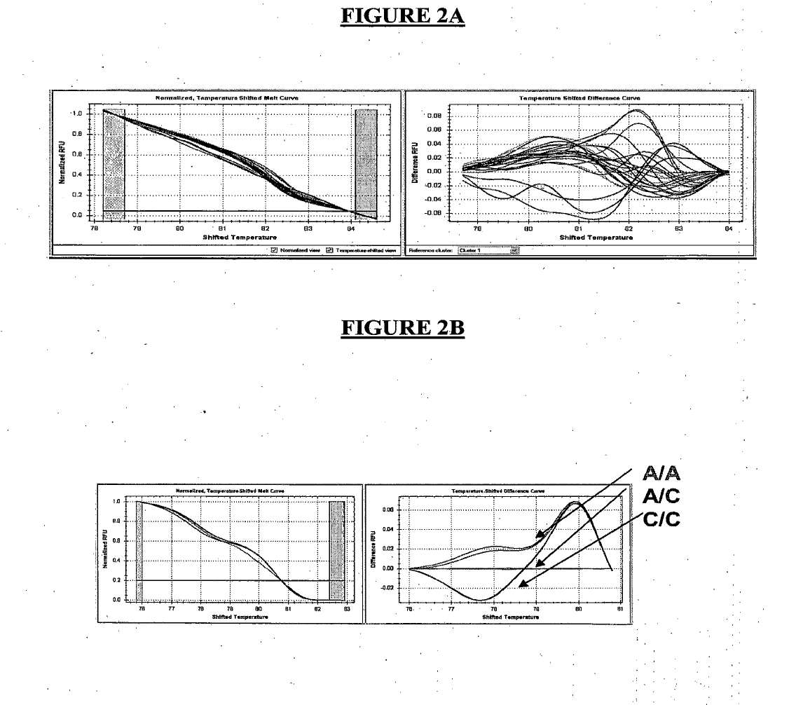

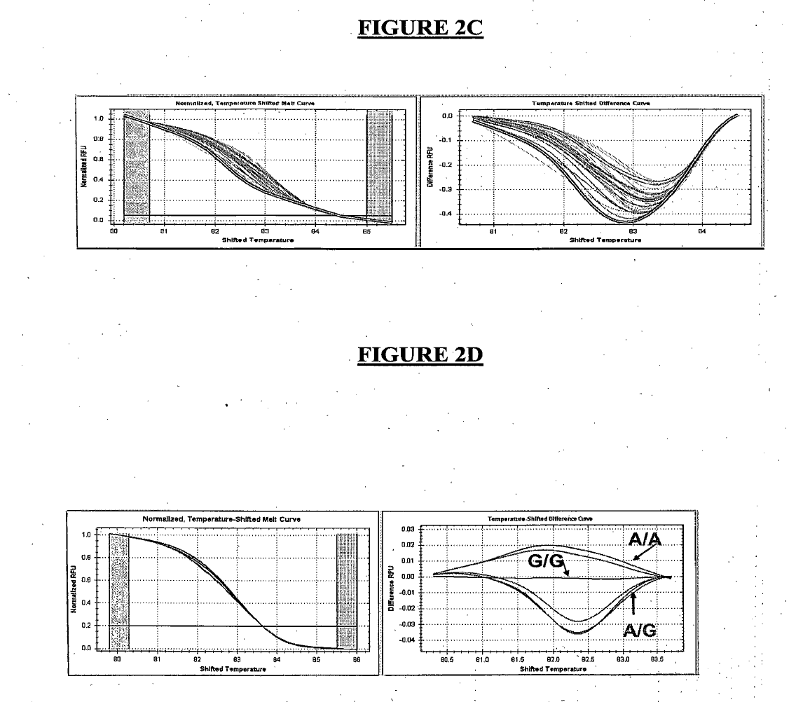

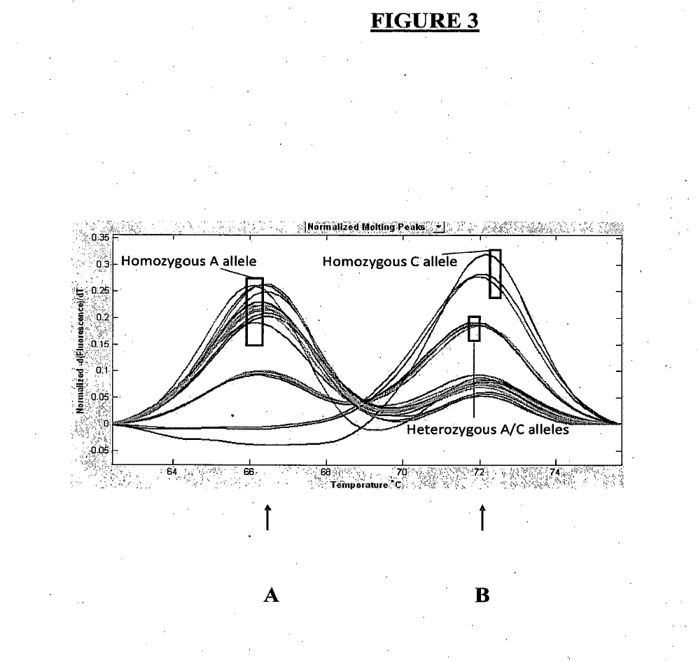

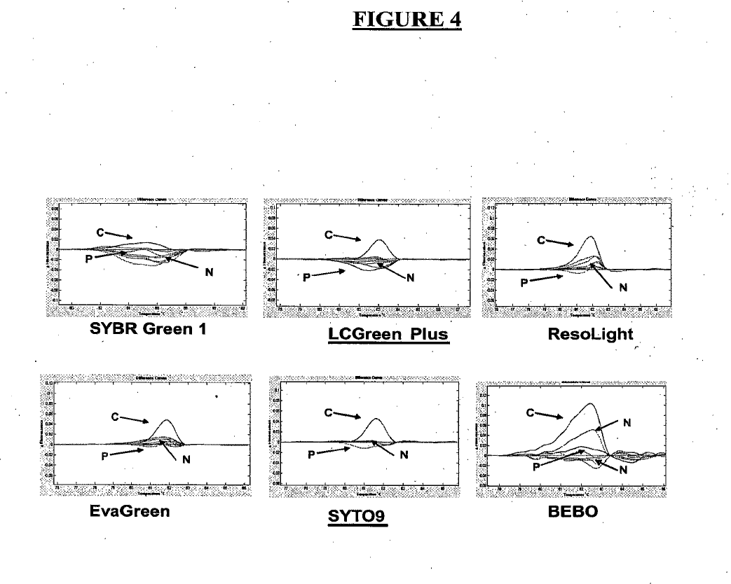

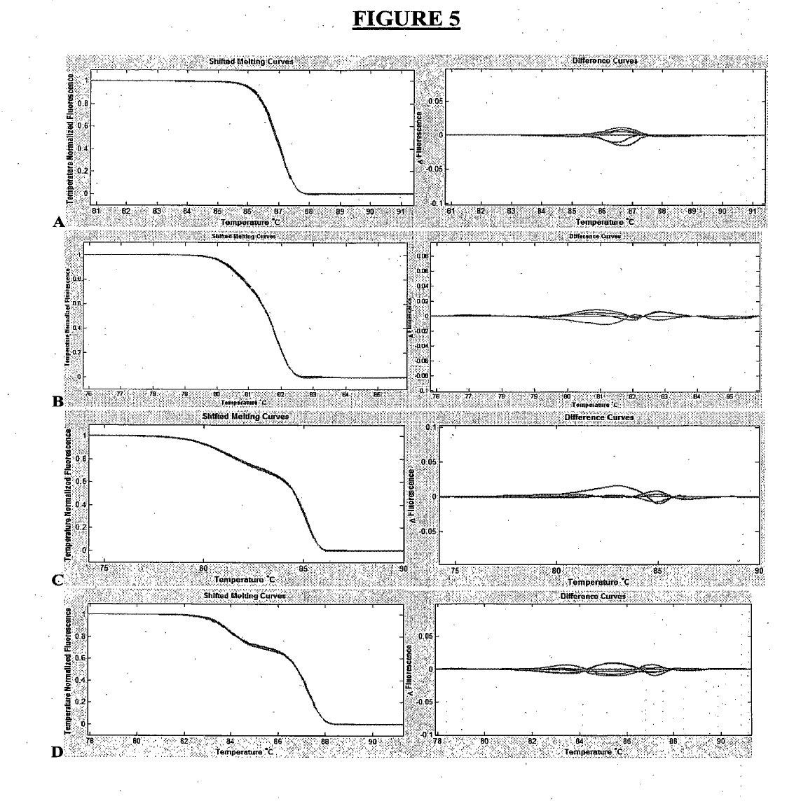

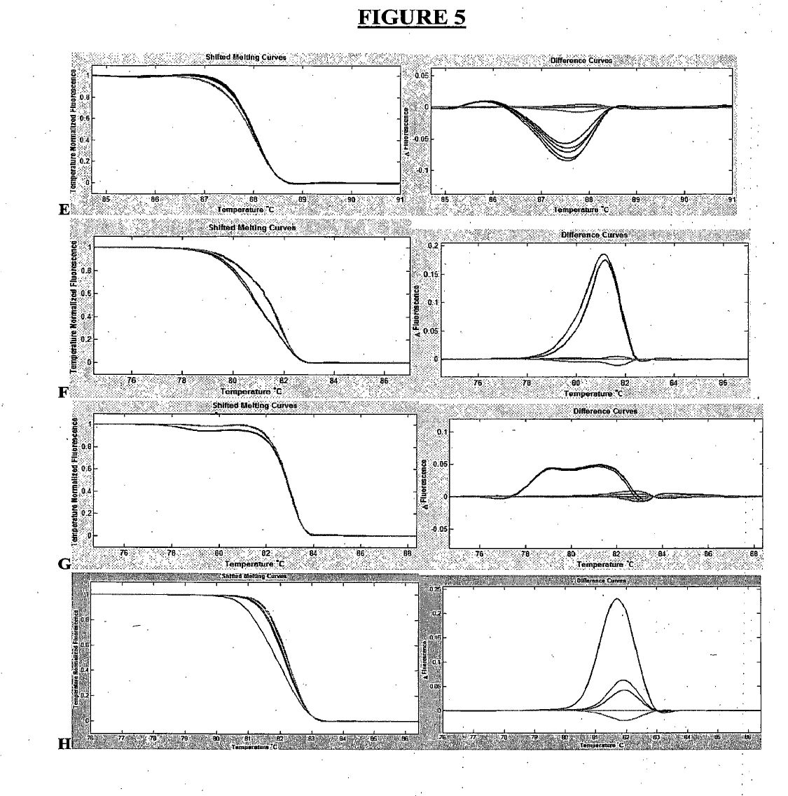

### DETAILED DISCLOSURE

According to the invention there is provided a method of determining sequence variants, including mutations and polymorphisms within a large gene preferably within the DMD gene, contributing to disease, preferably muscle disease comprising the steps of: (a) making a Whole-Genome Amplification (WGA) step to obtain sufficient amounts of genetic templates for DNA analysis. (b) enriching the WGA sample with nested primers designed for the large gene; (c) using the enriched WGA sample for high resolution melt (HRM); and (d) detecting differential melt profiles during the transition from double strand to single strand with an increase in temperature wherein sequence point mutations within the gene affects the thermal stability and gives a different melt profile from the normal non-mutated gene sequence. The method may further comprise the step of spiking the DNA being screened with DNA from a phenotypically normal individual in order to induce synthetic heterozygosity. The method in parts (c) and (d) may also work directly on genomic samples without WGA step if sufficient DNA material is present.

The method involves the melting of double stranded DNA into single stranded DNA with increasing temperature such that amplicons with sequence differences will generate different melt profiles due to the difference in thermal stability. These melt profiles are detected-due to the use of saturating intercalating DNA dyes which are released as the DNA strands melt and are captured by new generation melt instruments which are capable of much higher data density capture than conventional real-time PCR machines. This method is thus also called high resolution melting (HRM) analysis.

The HRM step may include a cleaning step such as the use of high magnesium concentration, exo-nuclease digestion, dephosphorylation treatment or filtration steps or any other method known in the art that could adequately clean the enriched WGA sample. Four mutations in the DMD gene were amplified using multiple displacement amplification (MDA) and analyzed by HRM. Melting patterns of WGA and genomic DNA were concordant for 3 out of 4 mutations, and discordant results were eliminated after post-PCR dephosphorylation treatment was performed. This is the first known study showing the effect of magnesium salt concentrations and dephosphorylation treatment on melt-analysis using WGA samples. Additionally, use of an additional nested PCR step for enriching the WGA products has been shown to improve the melt analysis and differentiate the different genotypes of mutation present in a sample. Use of WGA samples for melt-analysis is useful for genotyping/mutation screening in samples of limiting DNA templates such as in prenatal diagnosis, embryo pre-implantation diagnosis, and other such methods.

This method allows for quick screening of both known and unknown mutations as primers can be designed to optimally amplify regions simultaneously at same or similar sets of conditions for the entire gene, and the amplified products melted immediately after the PCR process to generate the melt profiles within 1 or 2 minutes either in a single closed tube or plate-based or chip-based microfluidics approach without a need for further laboratory manipulation. This reduces external contamination as well as reduces the number of steps for analysis. Following this primary screen, only the candidate sequence region showing abnormal melt-profiles (as compared with controls) need to be further analysed by sequencing. This reduces the need to sequence all the 79 exons per patient (which actually involve sequencing of more than 100 sequence regions as some exons are too large or contain repeat sequences to allow amplification or sequencing in a single reaction assay per exon).

For genotyping of known mutations, carrier screening by testing for absence or presence of the variant can similarly be quickly done on large number of samples by merely comparing the melt-profiles that are generated.

For some SNPs or point mutations, the discrimination in melt profiles between different hemizygous or homozygous alleles by HRM may be insufficient for clear detection. Spiking the test DNA with DNA from a phenotypically normal individual in order to induce synthetic heterozygosity may increase differentiation and allow easier calling of screening and genotyping melt profile results. DNA from a phenotypically normal individual is a nucleic acid sequence having very high level of homology with a known normal gene sequence of the gene of interest. In a preferred embodiment the DNA from a phenotypically normal individual is a nucleic acid sequence having very high level of homology with SEQ ID. NO. 1. The nucleic acid is taken from an individual who shows no phenotypic symptoms of a disease known to be caused when there are mutations present in the gene of interest. In a preferred embodiment the nucleic acid is taken from an individual who shows no phenotypic symptoms of a DMD or BMD known to be caused when there are mutations present in the DMD gene of SEQ ID. NO. 1.

“Spiking” of a DNA sample describes the mixing of the DNA sample to be tested with another DNA sample from a phenotypically normal donor. The genotype of the donor or spiking DNA is preferably known, though the process will also work should it be unknown. The effect of having two non-identical copies of a specific gene in a single solution is to create an artificial heterozygote. This is important for hemizygotic genes such as the DMD Gene and other X-linked genes on the sex chromosomes as they occur in only 1 copy in male individuals. For female samples, the necessity of spiking is diminished but it can still be performed. In HRM assays, the presence of 2 amplicons differing in sequence by only 1 base (as in the example of a single base mutation in the patient which is not present in the spiking/donor DNA) would result in not only the presence of perfectly-matched double-stranded DNA, but also the presence of double-stranded DNA with mismatches. This combination will significantly alter the melt profile of the sample, and explains why heterozygotes always become easily distinguishable from homozygotes in HRM, regardless of whether both homozygotes show sufficient differentiation between themselves. The optimal copy number spiking ratio is 1:1, though other ratios will also result in increased differentiation and changed melt profiles. Spiking is useful in detecting point mutations or SNPs which would otherwise have extremely subtle effects, and can be performed prior to PCR by mixing the 2 DNA templates, or post-PCR by mixing the PCR products.

In some regions of the gene, the presence of polymorphisms or repeat sequences may generate complex profiles which are difficult to analyse or interpret. For such regions, we have designed probes to hybridize to these complex regions such that a nested PCR is carried out with the second PCR generating an asymmetric product with more copies of the strand to which the probe will bind. The probe is designed to target the complex sequence but with a Tₘ that can generate a different temperature window upon melting. Hence, the probe-amplicon duplex and the whole amplicon duplex generates different melt-curves at two different distinct temperature windows allowing interrogation of both the region of polymorphism as well as mutation screening in the larger amplicon. The probe can be detected without being labelled but is blocked at its 3′ end to prevent extension of the oligonucleotides during PCR elongation step.

This method is simple, rapid and cheap. It requires only a PCR reaction, dye and melting equipment capable of capturing and generating the different melt profiles. It does not need real-time amplification and is performed within 1-2 minutes after PCR in a closed tube homogeneous assay. It does not require expensive labelled probes or extensive technical time for analysis of the results. As a clinical assay, it is affordable to patients. It can be customized as a commercial kit based assay-system offering diagnosis for entire gene. It has practical applications in detection of muscle diseases such as DMD and BMD due to the large size of the gene and the considerable cost involved if more than 100 PCR and sequencing reactions have to be carried out per patients It is also a rapid method with short turn-around time as only a PCR amplification and melt step is required. This method makes genetic testing more affordable for patients. It will contribute towards not only confirming molecular diagnosis but also for differential diagnosis.

### Whole Genome Amplification

Whole Genome Amplification (WGA): may be a technique designed to amplify all or some of the DNA in a sample. Several whole-genome amplification (WGA) techniques have been presented as capable of amplifying DNA from trace quantities and with less error than traditional PCR. Some preferred methods to perform WGA may include multiple-displacement amplification (MDA), that uses the highly processive p29 DNA polymerase and random exonuclease-resistant primers in an isothermal amplification reaction. This method is based on strand-displacement synthesis there are commercially available MDA kits such as GE MDA kit and the Sigma WGA kit. Some other methods may include PCR-based methods for WGA including degenerated oligonucleotide primed PCR (DOP-PCR) and primer extension PCR and ligation mediated PCR (LM-PCR). WGA kit (Sigma): may be a commercially available kit for conducting whole genome amplification. A new WGA method known as OmniPlex, converts randomly fragmented genomic DNA into a library of inherently amplifiable DNA fragments of defined size. This library can be effectively amplified several thousand fold with the help of a high-fidelity DNA polymerase. The library can be re-amplified to achieve a final amplification of over a million fold without degradation of representation. Similarly any WGA known in the art may be suitable for the invention.

Potential advantages of WGA include amplifying DNA from nanogram amounts, and large cost and time savings compared with alternatives such as generating cell lines from individuals. Typically between 0.1 to 10 ng of starting genomic DNA can be used for WGA, and after the WGA treatment, between 0.1 ng to 10 ng of the products can be used for the melt analysis.

### Probes and Primers for Mutation Screening in DMD/BMD

The probes and primers used for mutation screening and genotyping in DMD/BMD are targeted to specific sequences and uniquely designed so that they can be amplified and melted at universal sets of conditions and temperatures to reduce the number of different PCR assays required to screen the entire gene simultaneously. Nested primers are designed to reduce the contamination in products due to the amplification of unexpected primer binding sites effectively enriching the samples for use in the HRM ensuring very clean melt profiles that allow a quick visual detection of any mutation in a particular amplicon.

Polymerase chain reaction (PCR) itself is the process used to amplify nucleic acid samples. Conventional PCR requires primers complementary to the termini of the target nucleic acid. A problem with primers is that they often bind to incorrect regions of the nucleic acid, giving unexpected products. Nested primers comprise two sets of primers, used in two successive runs of polymerase chain reaction, the second set intended to amplify a secondary target within the first run product or a sequence that binds to a segment of the first amplicon to ensure only the intended locus is examined in the HRM. It is very unlikely that any of the unwanted PCR products contain binding sites for both the new primers, ensuring the product from the second PCR has little contamination from unwanted products of primer dimers, hairpins, and alternative primer target sequences.

The product from the first reaction undergoes a second run with the second set of primers. This requires unique knowledge of various factors such as melt-domains, melting temperatures, sequence specific properties and experimental parameters affecting melt-profiles. This methodology can be applied for interrogation of any sets of genes which can be carried out under similar assay conditions.

The outer primers (first primer set) are designed based on coverage of the sequences amplified by the inner primers (second primer set). All the inner primers are similar to those for the normal melt analysis, and for the WGA enrichment method, outer primers are designed such that primer melting temperatures (Tm) range from 56-64° C. and the maximum difference between the Tm of each primer in a pair should not exceed 1° C. Primer GC Content range from 10-90%. Primer Sizes range from 17-30 bases. Maximum Self-Complementarity is set at 6, while Maximum 3′ Stability set to 8.

“Probes”. Polynucleotide polymorphisms associated with DMD alleles are detected by hybridisation with a polynucleotide probe which forms a stable hybrid with that of the target sequence, under stringent to moderately stringent hybridisation and wash conditions. If it is expected that the probes will be perfectly complementary to the target sequence, stringent conditions will be used. Hybridisation stringency may be lessened if some mismatching is expected, for example, if variants are expected with the result that the probe will not be completely complementary. Conditions are chosen which rule out nonspecific/adventitious bindings, that is, which minimize noise. Since such indications identify neutral DNA polymorphisms as well as mutations, these indications need further analysis to demonstrate detection of a DMD in muscle disease such as DMD and BMD.

Probes for DMD nucleic acid may be derived from the sequences of the DMD gene or its cDNAs. The probes may be of any suitable length, which span all or a portion of an exon or intron of the DMD gene and which allow specific hybridisation to the DMD gene. If the target sequence contains a sequence identical to that of the probe, the probes may be short, e.g., in the range of about 8-30 base pairs, since the hybrid will be relatively stable under even stringent conditions. If some degree of mismatch is expected with the probe, i.e., if it is suspected that the probe will hybridize to a variant region, a longer probe may be employed which hybridises to the target sequence with the requisite specificity.

The probes will include an isolated polynucleotide attached to a label or reporter molecule and may be used to isolate other polynucleotide sequences, having sequence similarity by standard methods. For techniques for preparing and labeling probes see, e.g Sambrook et al., 1989: “Molecular Cloning: a laboratory manual. Sambrook, J., Fritsch, E. F. and Maniatis, T. (1989). Coldspring Harbour Laboratory Press, Coldspring Harbour, N.Y. Other similar polynucleotides may be selected by using homologous polynucleotides. Alternatively, polynucleotides encoding these or similar polypeptides may be synthesized or selected by use of the redundancy in the genetic code. Various codon substitutions may be introduced, e.g., by silent changes (thereby producing various restriction sites) or to optimize expression for a particular system. Mutations may be introduced to modify the properties of the polypeptide, perhaps to change ligand-binding affinities, interchain affinities, or the polypeptide degradation or turnover rate.

Probes comprising synthetic oligonucleotides or other polynucleotides of the present invention may be derived from naturally occurring or recombinant single- or double-stranded polynucleotides, or be chemically synthesized. Probes may also be labeled by nick translation, Klenow fill-in reaction, or other methods known in the art.

Portions of the polynucleotide sequence having at least about eight nucleotides, usually at least about 15 nucleotides, and fewer than about 6 kb, usually fewer than about 1.0 kb, from a polynucleotide sequence encoding dystrophin protein are preferred as probes. The probes may also be used to determine whether mRNA encoding dystrophin protein is present in a cell or tissue.

The present invention provides one or more DMD polynucleotides or fragments thereof comprising mutations with respect to the wild type sequence, such as the sequence shown in SEQ ID No. 1. In a further embodiment, the present invention provides a plurality of DMD polynucleotides or fragments thereof for use in screening the DNA of an individual for the presence of one or more mutations/polymorphisms. The plurality of sequences is conveniently provided immobilized to a solid substrate as is described below.

### High Resolution Melt

Method includes purification and enrichment of products for melt analysis. It employs techniques to amplify single product at high efficiency using optimized conditions with Mg salt concentrations; and/or treatment with exonuclease I amd shrimp alkaline phosphatase enzymes to remove primers and nucleotides from post-PCR products prior to melt; and/or ethanol precipitation of WGA products; and/or enrichment of the primary WGA product by a nested amplification step using outer primer pairs in first round PCR followed by inner primer pairs in second round PCR as a step before the final melt analysis. High Resolution Melt or HRM analysis as it will be referred to herein is a technique for the detection of mutations, polymorphisms and epigenetic differences in double stranded DNA samples. HRM analysis is performed on double stranded DNA samples. Typically the user will use polymerase chain reation (PCR) prior to HRM analysis to amplify the DNA region in which their mutation of interest lies. Essentially the PCR process turns a tiny amount of the region of interest in the DNA into a large amount, so you have quantities large enough for better analysis. In the tube there are now many of copies of the region of DNA of interest. This region that is amplified is known as the amplicon. After the PCR process the HRM analysis begins. The process is simply a precise heating of the amplicon DNA from around 50° C. up to around 95° C. At some point during this process, the melting temperature of the amplicon is reached and the two strands of DNA separate or “melt” apart. HRM is monitored by using a fluorescent dye. The dyes that are used for HRM are known as intercalating dyes and have a unique property. They bind specifically to double-stranded DNA and when they are bound they fluoresce brightly. As the type of dyes used are fully saturating, they will bind at all possible positions on the double stranded DNA such that the strands are fully statured with the dye. In the absence of double stranded DNA they have nothing to bind to and they only fluoresce at a low level. At the beginning of the HRM analysis there is a high level of fluorescence in the sample because of the billions of copies of the amplicon. But as the sample is heated up and the two strands of the DNA melt apart, presence of double stranded DNA decreases and thus fluorescence is reduced. The HRM machine has a camera that monitors and captures this process by measuring the fluorescence. The machine then simply plots this data as a graph known as a melt curve, showing the level of fluorescence vs the temperature. The melting temperature of the amplicon at which the two DNA strands come apart is entirely predictable. It is dependent on the sequence of the DNA bases. If two samples from two different people are compared, they should give exactly the same shaped melt curve or profile. However if one of the individual has a mutation in the DNA region that was amplified, then this will alter the temperature at which the DNA strands melt apart. So now the two melt curves appear different. The difference may only be subtle, perhaps a fraction of a degree, but because the HRM machine has the ability to monitor this process in “high resolution”, it is possible to accurately document these changes and therefore identify if a mutation is present or not.

The present invention relates to a fluorescence-based method which is able to determine whether a DNA sample is identical to another DNA sample in DNA sequence. This method is sufficiently sensitive to detect a difference of a single nucleotide base pair in DNA fragments of up to 250 bases in length. This method utilizes fluorescence-monitoring of the gradual and progressive thermal denaturation of double-stranded DNA (dsDNA) of various origins and configurations. Fluorescence is provided by fluorescent dsDNA-binding dyes which fluoresce while incorporated into dsDNA. As the dsDNA denatures, dye molecules are released into the solution and cease to fluoresce. Sufficient amount of the sample DNA for screening may be produced by various Polymerase Chain Reaction (PCR) methods as well as various Whole Genome Amplification (WGA) methods. A wild-type DNA fragment may be used as a control for comparison in mutation scanning applications.

One preferred embodiment of the invention is for the detection of variants (including polymorphisms and mutations) in the human DMD gene, though the method can be applied to any gene in any organism of known genome.

An isolated DMD nucleic acid molecule is disclosed which molecule typically encodes a dystrophin polypeptide, allelic variant, or analog, including fragments, thereof. Specifically provided are DNA molecules selected from the group consisting of: (a) DNA molecules set out in SEQ ID NO: 1, or fragments thereof; (b) DNA molecules that hybridize to the DNA molecules defined in (a) or hybridisable fragments thereof; and (c) DNA molecules that code an expression for the amino acid sequence encoded by any of the foregoing DNA molecules.

Preferred DNA molecules according to the invention include DNA molecules comprising the sequence set out in SEQ ID NO: 1 or fragments thereof.

A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced therefrom.

An “isolated” or “substantially pure” nucleic acid (e.g., an RNA, DNA or a mixed polymer) is one which is substantially separated from other cellular components which naturally accompany a native human sequence or protein, e.g., ribosomes, polymerases, many other human genome sequences and proteins. The term embraces a nucleic acid sequence or protein that has been removed from its naturally occurring environment, and includes recombinant or cloned DNA isolates and chemically synthesized analogs or analogs biologically synthesized by heterologous systems.

“DMD Allele” refers to normal alleles of the DMD gene sequence as well as alleles carrying variations.

“DMD gene sequence,” “DMD gene,” “DMD nucleic acids” or “DMD polynucleotide” each refer to polynucleotides that are likely to be expressed in muscle tissue. Mutations at the DMD gene sequence may be involved in muscle disease. Mutation nomenclature uses the NM_{—}004006.2 version of the muscle cDNA transcript for the human DMD gene (also known as M-dystrophin) set out in SEQ ID. No 1. The genomic sequence reference number for the primers which covers flanking intronic and/or branchpoint sequences uses Homo sapiens DMD refseq (NCBI) NG_{—}012232.1 covering sections of sequence SEQ ID No. 1.

The DMD gene sequence is intended to include coding sequences, intervening sequences and regulatory elements controlling transcription and/or translation. The DMD gene sequence is intended to include all allelic variations of the DNA sequence. The mRNA and coding sequences cover those from all known transcripts and isoforms of the DMD gene, such as M-dystrohpin (NM_{—}004006.2), L-dystrophin (NM_{—}004007.2), C-dystrophin (NM_{—}000109.3), P1-dystrophin (NM_{—}004009.3), P2-dystrophin (NM_{—}004010.3), etc.

These terms, when applied to a nucleic acid, refer to a nucleic acid that encodes a DMD polypeptide, fragment, homologue or variant, including, e.g., protein fusions or deletions. The nucleic acids of the present invention will possess a sequence that is either derived from, or substantially similar to a natural DMD encoding gene or one having substantial homology with a natural DMD encoding gene or a portion thereof. The coding sequence for human DMD polynucleotide is shown in SEQ ID NO: 1, with the amino acid sequence shown in SEQ ID NO: 2.

A nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases.

Alternatively, substantial homology or (identity) exists when a nucleic acid or fragment thereof will hybridise to another nucleic acid (or a complementary strand thereof) under selective hybridisation conditions, to a strand, or to its complement. Selectivity of hybridisation exists when hybridisation that is substantially more selective than total lack of specificity occurs. Typically, selective hybridisation will occur when there is at least about 55% identity over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Thus, polynucleotides of the invention preferably have at least 75%, more preferably at least 85%, more preferably at least 90% homology to the sequences shown in the sequence listings herein. More preferably there is at least 95%, more preferably at least 98%, homology. Nucleotide homology comparisons may be conducted as described below for polypeptides. A preferred sequence comparison program is the GCG Wisconsin Bestht program described below. The default scoring matrix has a match value of 10 for each identical nucleotide and −9 for each mismatch. The default gap creation penalty is −50 and the default gap extension penalty is −3 for each nucleotide.

In the context of the present invention, a homologous sequence is taken to include a nucleotide sequence which is at least 60, 70, 80 or 90% identical, preferably at least 95 or 98% identical at the amino acid level over at least 20, 50, 100, 200, 300, 500 or 1000 nucleotides with the nucleotides sequences set out in SEQ ID. No 1. In particular, homology should typically be considered with respect to those regions of the sequence that encode contiguous amino acid sequences known to be essential for the function of the protein rather than non-essential neighbouring sequences. Preferred polypeptides of the invention comprise a contiguous sequence having greater than 50, 60 or 70% homology, more preferably greater than 80, 90, 95 or 97% homology, to one or more of the nucleotides sequences of SEQ ID NO: 1 from 1 to 13993, which encode amino acids 1 to 3685 of SEQ ID NO:2. Preferred polynucleotides may alternatively or in addition comprise a contiguous sequence having greater than 80, 90, 95 or 97% homology to the sequence of SEQ ID NO: 1 that encodes amino acids 1 to 3685 of SEQ ID NO:2.

Other preferred polynucleotides comprise a contiguous sequence having greater than 40, 50, 60, or 70% homology, more preferably greater than 80, 90, 95 or 97% homology to the sequence of SEQ ID NO: 1 that encodes amino acids 1 to 3685.

Nucleotide sequences are preferably at least 15 nucleotides in length, more preferably at least 20, 30, 40, 50, 100 or 200 nucleotides in length.

Generally, the shorter the length of the polynucleotide, the greater the homology required to obtain selective hybridization. Consequently, where a polynucleotide of the invention consists of less than about 30 nucleotides, it is preferred that the % identity is greater than 75%, preferably greater than 90% or 95% compared with the DMD nucleotide sequences set out in the sequence listings herein. Conversely, where a polynucleotide of the invention consists of, for example, greater than 50 or 100 nucleotides, the % identity compared with the DMD nucleotide sequences set out in the sequence listings herein may be lower, for example greater than 50%, preferably greater than 60 or 75%.

The main approach for this set of assays is the use of primers and probes to encompass coding exons, flanking intronic splice sites and branch-points, upstream promoter regions and downstream regulatory regions at the 3′ untranslated region of the DMD gene. A pair of primers were used for the amplification and melt assay of the regions with no known SNPs while additionally a set of probes and primers were used for regions with known presence of SNPs (as provided by dbSNP in NCBI database). Primers were designed based on the following features:

Primer Melting Temperatures (Tₘ) range from 56-64° C. in 2° C. increments. Maximum Difference between the Tₘ of each primer in a pair should not exceed 1° C. Primer GC Content range from 10-90%. Primer Sizes range from 17-30 bases. Maximum Self-Complementarity set at 6, while Maximum 3′ Stability set to 8. Product Sizes range from 100-250 bases in length, though whenever possible the amplicon size range should be 150-200 bases long. Whenever possible, primers for the individual exons encompass the predicted branching points for the exon and at least 5 bases of the following intron at the 3′ end. Due to the relatively small amplicon sizes, many exons require 2 or more overlapping amplicons for complete coverage of the branch points, exon, and 3′ flank. Primer sequences require checking by BLAST or other similar programmes to rule out the possibility of multiple binding sites. Where possible the primers were designed to amplify amplicons with only one melting domain. Primers are also designed to generate amplicons with ideally one melting domain and avoiding secondary structures, where possible. The designing of primers for this invention can be performed using Primer3 or other primer design software, structure prediction softwares.

Probes were designed based on the following features: A list of known Single Nucleotide Polymorphisms (SNPs) in the exonic regions can be obtained from online databases such as dbSNP. To interrogate exons containing SNPs, primers are designed by placement directly on the SNP followed by use of probe, or by generating amplicons to avoid the SNP region entirely. Coverage of the SNP regions can be obtained by the use of unlabelled hybridization probes which are modified at the 3′ end to prevent PCR extension. Hybridization probes are short fragments of DNA complementary to one strand of the DNA duplex. The probe works by effectively shortening the region of interrogation to the length of the probe, thereby magnifying any existing sequence difference. An important criteria in probe design is to have the SNP fall roughly in the middle of the probe as the further from the centre of the probe a SNP is located, the higher the probability the probe will fail to work as intended. The probes can be designed by Primer3 or any other primer design or probe selection software. Parameters for probe selection in Primer3 can be set. Hybridization Oligo Size range from 20-40 bases. Hybridization Oligo Tₘ range from 50-65° C., as it is vital that there be at least 10° C. between the denaturation temperature of the probe with that of the full-length PCR product. Fluorescent-labeled probes can also be designed using the same parameters and software, provided the excitation wavelength of the fluorescent label is close to the emission wavelength of the dsDNA-binding dye and the detector is capable of reading the excitation wavelength of the fluorescent label.

The term “polypeptide” refers to a polymer of amino acids and its equivalent and does not refer to a specific length of the product; thus, peptides, oligopeptides and proteins are included within the definition of a polypeptide. This term also does not refer to, or exclude modifications of the polypeptide, for example, glycosylations, acetylations, phosphorylations, and the like. Included within the definition are, for example, polypeptides containing one or more analogs of an amino acid (including, for example, natural amino acids, etc.), polypeptides with substituted linkages as well as other modifications known in the art, both naturally and non-naturally occurring.

In the context of the present invention, a homologous sequence is taken to include an amino acid sequence which is at least 60, 70, 80 or 90% identical, preferably at least 95 or 98% identical at the amino acid level over at least 20, 50, 100, 200, 300 or 400 amino acids with the amino acid sequence set out in SEQ ID. No 2. In particular, homology should typically be considered with respect to those regions of the sequence known to be essential for the function of the protein rather than non-essential neighbouring sequences. Preferred polypeptides of the invention comprise a contiguous sequence having greater than 50, 60 or 70% homology, more preferably greater than 80 or 90% homology, to one or more of amino acids of SEQ ID NO: 2.

Other preferred polypeptides comprise a contiguous sequence having greater than 40, 50, 60, or 70% homology, of SEQ ID No: 2. Although homology can also be considered in terms of similarity (i.e. amino acid residues having similar chemical properties/functions), in the context of the present invention it is preferred to express homology in terms of sequence identity. The terms “substantial homology” or “substantial identity”, when referring to polypeptides, indicate that the polypeptide or protein in question exhibits at least about 70% identity with an entire naturally-occurring protein or a portion thereof, usually at least about 80% identity, and preferably at least about 90 or 95% identity.

Homology comparisons can be conducted by eye, or more usually, with the aid of readily available sequence comparison programs. These commercially available computer programs can calculate % homology between two or more sequences.

Percentage (%) homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG. Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software that can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all- or -nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

“dystrophin protein” or “DMD polypeptide” refers to a protein or polypeptide encoded by the DMD gene sequence, variants or fragments thereof. Also included are proteins encoded by DNA that hybridize under high or low stringency conditions, to DMD encoding nucleic acids and closely related polypeptides or proteins retrieved by antisera to the dystrophin protein(s).

A polypeptide “fragment,” “portion” or “segment” is a stretch of amino acid residues of at least about five to seven contiguous amino acids, often at least about seven to nine contiguous amino acids, typically at least about nine to 13 contiguous amino acids and, most preferably, at least about 20 to 30 or more contiguous amino acids.

“Spiking” of a DNA sample describes the mixing of the DNA sample to be tested with another DNA sample from a phenotypically normal donor. The genotype of the donor or spiking DNA is preferably known, though the process will also work should it be unknown. The effect of having two non-identical copies of a specific gene in a single solution is to create an artificial heterozygote. This is important for hemizygotic genes such as the DMD Gene and other genes on the sex chromosomes as they occur in only 1 copy in male individuals. For female samples, the necessity of spiking is diminished but it can still be performed. In HRM assays, the presence of 2 amplicons differing in sequence by only 1 base (as in the example of a single base mutation in the patient which is not present in the spiking/donor DNA) would result in not only the presence of perfectly-matched double-stranded DNA, but also the presence of double-stranded DNA with mismatches. This combination will significantly alter the melt profile of the sample, and explains why heterozygotes will become more distinguishable from homozygotes in HRM, regardless of whether both homozygotes show sufficient differentiation between themselves. The optimal copy number spiking ratio is 1:1, although other ratios will also result in increased differentiation and change in melt profiles. Spiking is useful in detecting point mutations or SNPs which would otherwise show extremely subtle effects which may not be clearly detectable in the melt profiles, and can be performed prior to PCR by mixing the 2 DNA templates, or post-PCR by mixing the PCR products.

A phenotypically normal donor is a person who has no known symptoms of a disease known to be caused by the gene of interest.

Any DMD nucleic acid specimen, in purified or non-purified form, can be utilised as the starting nucleic acid or acids.

PCR is one such process that may be used to amplify DMD gene sequences. This technique may amplify, for example, DNA, cDNA or RNA, including messenger RNA, wherein DNA or RNA may be single stranded or double stranded. In the event that RNA is to be used as a template, enzymes, and/or conditions optimal for reverse transcribing the template to DNA would be utilized. In addition, a DNA-RNA hybrid that contains one strand of each may be utilized. A mixture of nucleic acids may also be, employed, or the nucleic acids produced in a previous amplification reaction described herein, using the same or different primers may be so utilised.

The specific nucleic acid sequence to be amplified, i.e., the polymorphic gene sequence, may be a fraction of a larger molecule or can be present initially as a discrete molecule, so that the specific sequence constitutes the entire nucleic acid. It is not necessary that the sequence to be amplified is present initially in a pure form; it may be a minor fraction of a complex mixture, such as contained in whole human DNA.

DNA utilized herein may be extracted from a body sample, such as blood, buccal cells, saliva, sera, tissue material, muscle tissue and the like by a variety of techniques. If the extracted sample has not been purified, it may be treated before amplification with an amount of a reagent effective to open the cells, or animal cell membranes of the sample, and to expose and/or separate the strand(s) of the nucleic acid(s). This lysing and nucleic acid denaturing step to expose and separate the strands will allow amplification to occur much more readily.

The deoxyribonucleotide triphosphates dATP, dCTP, dGTP and dTTP are added to the synthesis mixture, either separately or together with the primers, in adequate amounts and the resulting solution is heated to about 90 degrees-100 degrees C. from about 1 to 10 minutes, preferably from 1 to 4 minutes. After this heating period, the solution is allowed to cool, which is preferable for the primer hybridization. To the cooled mixture is added an appropriate agent for effecting the primer extension reaction (called herein “agent for polymerization”), and the reaction is: allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40 degree C. Most conveniently the reaction occurs at room temperature.

Specific oligonucleotide primers derived from DMD gene sequence may be useful in determining whether a subject is at risk of suffering from the ailments described herein. Primers direct amplification of a target polynucleotide (eg DMD or any of other muscle gene like FKRP or DYSF or CAPN3 or LAMA2 or LMNA or SG or SGCB) prior to sequencing. Primers used in any diagnostic assays derived from the present invention should be of sufficient length and appropriate sequence to provide initiation of polyrmerisation. Environmental conditions conducive to synthesis include the presence of nucleoside triphosphates and an agent for polymerisation, such as DNA polymerase, and a suitable temperature and pH.

Primers of the invention are preferably single stranded for maximum efficiency in amplification, but may be double stranded. If double stranded, primers may be first treated to separate the strands before being used to prepare extension products. Primers should be sufficiently long to prime the synthesis of DMD gene extension products in the presence of the inducing agent for polymerization. The exact length of a primer will depend on many factors, including temperature, buffer, and nucleotide composition. Oligonucleotide primers will typically contain 12-20 or more nucleotides, although they may contain fewer nucleotides. Primers were designed to amplify amplicons covering coding exons, flanking intronic splice sites and branch-points, upstream promoter regions and downstream regulatory regions at the 3′ untranslated region of the gene. A pair of primers was used for the amplification and melt assay of each of the regions with no known SNPs while additional a set of probes and primers were used for regions with known presence of SNPs (as provided by dbSNP in NCBI database).

For regions with SNPs, a set of probes were designed based on the following features: A list of known Single Nucleotide Polymorphisms (SNPs) in the exonic regions can be obtained from online databases such as dbSNP. SNPs are avoided by placement of primers directly on the SNP or avoiding the SNP region entirely. Coverage of the SNP regions can be obtained by the use of unlabelled hybridization probes which are modified at the 3′ end to prevent PCR extension. Hybridization probes are short fragments of DNA complementary to one strand of the DNA duplex. The probe works by effectively shortening the region of interrogation to the length of the probe, thereby magnifying any existing sequence difference. An important criteria in probe design is to have the SNP fall roughly in the middle of the probe as the further from the centre of the probe a SNP is located, the higher the probability the probe will fail to work as intended. The probes can be designed by Primer3 or any other primer design or probe selection software. Parameters for probe selection in Primer3 can be set. Hybridization Oligo Size range from 20-40 bases. Hybridization Oligo Tm range from 50-65° C., as it is vital that there be at least 10° C. between the denaturation temperature of the probe with that of the full-length PCR product. For the best effect the size of the full-length amplicon should be between 100 and 200 bp, although this will also work for amplicons more than 200 bp in size. Fluorescent-labeled probes can also be designed using the same parameters and software, provided the excitation wavelength of the fluorescent label is close to the emission wavelength of the dsDNA-binding dye and the detector is capable of reading the excitation wavelength of the fluorescent label. Additionally a set of probes and primers were used for regions with known presence of SNPs

Nested PCR are designed using an outer pair and an inner pair of primers. The inner pair of primers are the same used for the melt analysis as shown in Table 1 while the outer primers are shown in Table 3. The outer primers are designed based on coverage of the sequences amplified by the inner primers. All the inner primers are similar to those for the normal melt analysis, and for the WGA enrichment method, outer primers are designed such that primer melting temperatures (Tm) range from 56-64° C. and the maximum difference between the Tm of each primer in a pair should not exceed 1° C. Primer GC Content range from 10-90%. Primer Sizes range from 17-30 bases. Maximum Self-Complementarity is set at 6, while Maximum 3′ Stability set to 8.

Primers that may be used in diagnostic assays derived from the present invention should be designed to be substantially complementary to each strand of the DMD genomic gene sequence. This means that the primers must be sufficiently complementary to hybridise with their respective strands under conditions that allow the agent for polymerisation to perform. In other words, the primers should have sufficient complementarity with the 5′ and 3′ sequences flanking the mutation to, hybridise therewith and permit amplification of the DMD genomic gene sequence.

Oligonucleotide primers of the invention employed in the PCR amplification process that is an enzymatic chain reaction that produces exponential quantities of DMD gene sequence relative to the number of reaction steps involved. Typically, one primer will be complementary to the negative (−) strand of the DMD gene sequence and the other is complementary to the positive (+) strand of the DMD gene sequence. Annealing the primers to denatured nucleic acid followed by extension with an enzyme, such as the large fragment of DNA polymerase I (Klenow) and nucleotides, results in newly synthesised + and − strands containing the target within a DMD gene sequence. Because these newly synthesized sequences are also templates, repeated cycles of denaturing, primer annealing, and extension results in exponential production of a region of the DMD gene sequence defined by the primers. The product of the chain reaction is a discreet nucleic acid duplex with termini corresponding to the ends of the specific primers employed.

Oligonucleotide primers may be prepared using any suitable method, such as conventional phosphotriester and phosphodiester methods or automated embodiments thereof. In one such automated embodiment, diethylphosphoramidites are used as starting materials and may be synthesized.

The agent for polymerisation may be any compound or system which will function to accomplish the synthesis of primer extension products, including enzymes. Suitable enzymes for this purpose include, for example, E. coli DNA polymerase I, Klenow fragment of E. coli DNA polymerase, polymerase muteins, reverse transcriptase, other enzymes, including heat-stable enzymes (ie, those enzymes which perform primer extension after being subjected to temperatures sufficiently elevated to cause denaturation), such as Taq polymerase. Suitable enzyme will facilitate combination of the nucleotides in the proper manner to form the primer extension products that are complementary to each DMD gene sequence nucleic acid strand. Generally, the synthesis will be initiated at the 3′ end of each primer and proceed in the 5′ direction along the template strand, until synthesis terminates, producing molecules of different lengths.

The newly synthesised DMD strand and its complementary nucleic acid strand will form a double-stranded molecule under hybridizing conditions described above and this hybrid is used in subsequent steps of the process. In the next step, the newly synthesized double-stranded molecule is subjected to HRM denaturing conditions using any of the procedures described above to provide heat melt profiles.

The steps of denaturing, annealing, and extension product synthesis can be repeated as often as needed to amplify the target polymorphic gene sequence nucleic acid sequence to the extent necessary for detection. The amount of the specific nucleic acid sequence produced will accumulate in an exponential fashion. Amplification may also be achieved via real time PCR as known in the art.

Preferably, the method of amplifying DMD is by PCR, as described herein or real time PCR and as is commonly used by those of ordinary skill in the art. Alternative methods of amplification have been described and can also be employed as long as the DMD gene sequence amplified by PCR using primers of the invention is similarly amplified by the alternative means. Such alternative amplification systems include but are not limited to self-sustained sequence replication, which begins with a short sequence of RNA of interest and a T7 promoter. Reverse transcriptase copies the RNA into cDNA and degrades the RNA, followed by reverse transcriptase polymerizing a second strand of DNA. Another nucleic acid amplification technique is nucleic acid sequence-based amplification (NASBA) which uses reverse transcription and T7 RNA polymerase and incorporates two primers to target its cycling scheme. NASBA can begin with either DNA or RNA and finish with either, and amplifies to 10⁸ copies within 60 to 90 minutes. Alternatively, nucleic acid can be amplified by ligation activated transcription (LAT). LAT works from a single-stranded template with a single primer that is partially single-stranded and partially double-stranded. Amplification is initiated by ligating a cDNA to the promoter oligonucleotide and within a few hours, amplification is 10⁸ to 10⁹ fold. The QB replicase system can be utilized by attaching an RNA sequence called MDV-1 to RNA complementary to a DNA sequence of interest. Upon mixing with a sample, the hybrid RNA finds its complement among the specimen's mRNAs and binds, activating the replicase to copy the tag-along sequence of interest. Another nucleic acid amplification technique, ligase chain reaction (LCR), works by using two differently labeled halves of a sequence of interest that are covalently bonded by ligase in the presence of the contiguous sequence in a sample, forming a new target. The repair chain reaction (RCR) nucleic acid amplification technique uses two complementary and target-specific oligonucleotide probe pairs, thermostable polymerase and ligase, and DNA nucleotides to geometrically amplify targeted sequences. A 2-base gap separates the oligonucleotide probe pairs, and the RCR fills and joins the gap, mimicking normal DNA repair. Nucleic acid amplification by strand displacement activation (SDA) utilizes a short primer containing a recognition site for hincII with short overhang on the 5′ end that binds to target DNA. A DNA polymerase fills in the part of the primer opposite the overhang with sulfur-containing adenine analogs. HincII is added but only cuts the unmodified DNA strand. A DNA polymerase that lacks 5′ exonuclease activity enters at the site of the nick and begins to polymerize, displacing the initial primer strand downstream and building a new one which serves as more primer. SDA produces greater than 10⁷-fold amplification in 2 hours at 37 degrees C. Unlike PCR and LCR, SDA does not require instrumented temperature cycling. Another amplification system useful in the method of the invention is the QB Replicase System. Although PCR is the preferred method of amplification if the invention, these other methods can also be used to amplify the JMJD6 or JMJD6 and estrogen receptor gene sequence as described in the method of the invention.

A “sample”, as used herein, refers to a biological sample obtained from body fluid or a tissue in the body, for example a biopsy. In a preferred embodiment the sample is will be a “clinical sample,” which is a sample derived from a patient such as a fine needle biopsy sample or blood extraction. A “sample” may also include a section of tissue such as a section taken from a frozen or fixed tissue. Tissue samples can be obtained from muscle cells.

### Diagnosis

Diagnostic and prognostic methods will generally be conducted using a biological sample obtained from either a pre-natal or pre embryo implantation source or from any individual undergoing screening of muscle disease such as DMD or BMD or the like. A “sample” refers to a sample of tissue or fluid suspected of containing an analyte polynucleotide or polypeptide from an individual including, but not limited to, e.g., plasma, serum, spinal fluid, lymph fluid, the external sections of the skin, respiratory, intestinal, and genitourinary tracts, tears, saliva, blood cells, organs, tissue including muscle tissue and samples of in vitro cell culture constituents.

According to the diagnostic and prognostic methods of the present invention, alteration of the DMD gene sequence when compared to the DMD gene sequence taken from a normal, sample may be detected using anyone of the methods described herein. In addition, the diagnostic and prognostic methods can be performed to detect the DMD gene sequence expression and confirm the presence or absence of a functional dystrophin protein.

### Detection Kits

Detection kits may comprise reagents for whole genome amplification, at least two nested primers specific to the gene of interest and reagents' for high resolution melt analysis. The kit may further contain one or more of the primers and probes of the invention, reagents for whole genome amplification, intercalating dyes, PCR reagents, cleaning or filtering reagents such as magnesium salts preferably MgCl₂ or physical filters as known in the art or any combination thereof. Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, screening for genetic mutations in large genes, specifically in screening for uncommon mutations. In one embodiment the screening is for mutation screening, genotyping and other applications.

### EXAMPLES OF PREFERRED EMBODIMENTS

### Protocol for Whole Genome Amplification of Genomic DNA Followed by Product Purification and Nested Primer Amplification for High Resolution Melting Applications

This protocol is for the whole genome amplification (WGA) of extracted genomic DNA using Multiple Displacement Amplification method, for example, with use of Repli-g Midi Kit (Qiagen) followed by WGA product purification using Exonuclease I-Shrimp Alkaline Phosphatase (ExoSAP) digestion, PureLink™ PCR Purification Kit (Invitrogen), or Nested Polymerase Chain Reaction (nPCR) prior to High Resolution Melting (HRM) with any of the methods incorporated as well.

For each reaction, 5 μl of 20 ng/μl DNA template used. This protocol is for a single reaction. Proportional scale-ups should be conducted for higher sample numbers.
Prepare Buffer D1 by diluting 0.7 μl of reconstituted Buffer DLB with 5 μl of sterile water.
Prepare Buffer N1 by diluting 1.1 μl of Stop Solution with 10.3 μl of sterile water.
Prepare Buffer DLB by adding 500 μl of sterile water to the tube.
Place the 5 μl of sample in a 1.5 ml Eppendorf tube and add 5 μl of Buffer D1.
Vortex briefly and pulse spin. Incubate at room temperature for 3 minutes.
Add 10 μl Buffer N1 to the sample. Vortex briefly and pulse spin.
Thaw Repli-g Polymerase on ice and all other components at room temperature. Vortex briefly and pulse spin.
Prepare Master Mix on ice by adding 1 μl of Repli-g Polymerase to 29 μl of Repli-g Reaction Buffer. Mix and pulse spin.
Add the 30 μl of Master Mix to the 20 μl of denatured DNA template.
Incubate at 30° C. for 10-16 hours.
Heat the sample to 65° C. for 3 minutes to denature the polymerase. WGA products can be stored frozen or refrigerated.

WGA products can be quantitated spectrometrically and diluted to suitable working concentrations. However, measured quantities may not be accurate due to difficulties in establishing a calibrating blank and the presence of nonspecific DNA products and various proteins.

PureLink™ PCR Purification Kit

This section of the protocol deals with using, the PureLink™ PCR Purification Kit to remove truncated and nonspecific WGA products and primer dimers by spin column techniques. Binding Buffer HC should be used as it will retain only DNA fragments larger than 600 bp after purification.

The recommended volume of product to be purified is 50-100 μl. WGA products should be diluted at least 2 times before being applied to the spin column to reduce the viscosity and to prevent overloading the column filter. This protocol is for a single reaction, based on the 250 reaction PureLink™ PCR Purification Kit. Proportional scale-ups should be conducted for higher sample numbers.

Prepare Binding Buffer HC by adding 11 ml isopropanol to 109 ml undiluted Binding Buffer HC.
Prepare Wash Buffer by adding 160 ml 96-100% ethanol to 40 ml undiluted Wash Buffer.
Mix 4 volumes of prepared Binding Buffer HC with 1 volume of WGA product in an appropriate sterile microfuge tube.
Pipette the mixture into the provided PureLink™ Spin Column in Collection Tube without touching the filter. Centrifuge at 10,000 g at room temperature for 60 seconds.
Discard the flowthrough liquid in the collection tube and briefly dry the collection tube by tapping it on a piece of clean absorbent paper. Reuse the collection tube for further steps.
Add 650 μl of prepared Wash Buffer to the spin column and centrifuge at 10,000 g at room temperature for 60 seconds.
Discard flowthrough and dry the collection tube. OPTIONAL: Repeat Steps 8 & 9.
Return collection tube to spin column and centrifuge the spin column at maximum speed for 3 minutes.

Discard collection tube and place spin column in provided sterile 1.7 ml PureLink™ Elution Tube.

Add 30 μl Elution Buffer or sterile water (pH 7-8) to the spin column and incubate at room temperature for 2 minutes.
Centrifuge for at maximum speed for 2 minutes at room temperature.
Repeat Step 12 using 20 μl of Elution Buffer or sterile water. The combined DNA-containing elute volume should be around 48 μl.
Purified WGA products can be quantitated spectrometrically and diluted to suitable working concentrations.

### Magnesium Salt Buffer Wash

This section of the protocol deals with removal of excess salt from PCR products and resuspension with magnesium salt buffer prior to melt analysis. PCR products are resuspended using 3.0 M sodium acetate (ph 5.2) and 70% ethanol. A stock solution of 450 mM MgCl₂ was prepared by dissolving 9.15 g magnesium chloride hexahydrate in 95.14 ml water. Buffer solutions containing 2.5 mM, 25 mM, 75 mM, 125 mM or 175 mM Mg Cl₂ was added to the PCR product and resuspended in a final volume of 10 ul with water.

### ExoSAP

This section of the protocol deals with using the Exonuclease I-Shrimp Alkaline Phosphatase enzymatic digestion to remove truncated and nonspecific WGA products and primer dimers. The protocol for 10 reactions is given for ease of calculation. For more or less reactions, appropriate scale-ups or scale-downs should be performed.

Prepare 6 μl of each WGA product to be purified in sterile microfuge tubes or PCR plates.
Prepare a master mix on ice in the following order:

| | |
|---|---|
| Sterile water | 38.1 μl |
| Exonuclease I (20,000 U/ml) | 0.7 μl |
| Shrimp Alkaline Phosphatase (1 U/μl) | 3.2 μl |
| Total | 42.0 μl |

Aliquot 4 μl of the master mix into each well or tube containing 6 μl of WGA product.
Incubate the reactions at 37° C. for 40 minutes, followed by an enzyme denaturation step at 85° C. for 10 minutes.
Purified WGA products can be quantitated spectrometrically and diluted to suitable working concentrations. However, measured quantities may not be accurate due to difficulties in establishing a calibrating blank and the presence of various proteins.

### Nested PCR

This section of the protocol deals with using nested PCR (nPCR) for the dilution of nonspecific WGA products and primer dimers during the PCR stage. For larger sample sizes, appropriate scale-ups should be performed. This protocol utilizes batches of 8 individual DNA samples (henceforth referred to as a ‘set’) per pair of primers. Each set serves as 1′HRM analyses unit.

An example is shown as follows:

Nested Primer Sequences.

### Exon 2:

### Outer Primers:

| F: | 5′- gcctggccatttttcaga -3′ |
|---|---|
| R: | 5′- atatttccagatttgcacagctaa -3′ |

### Inner Primers:

| | |
|---|---|
| F: | 5′- catcataatggaaagttactttg -3′ |
| R: | 5′- agatcttaaaagtaaagtaacaaacc -3′ |

### Exon 30:

### Outer Primers:

| | |
|---|---|
| F: | 5′- ggaagctgcgaaatctgtct -3′ |
| R: | 5′- atcaaaacaaccccatggaa -3′ |

### Inner Primers:

| | |
|---|---|
| F: | 5′- acatttaactgatacactcttattcc -3′ |
| R: | 5′- ACTGCTTGTCATGAATGTG -3′ |

### Exon 37:

### Outer Primers:

| | |
|---|---|
| F: | 5′- gcatgtgcttgctctcattt -3′ |
| R: | 5′- aaaccttgctgtggggtcta -3′ |

### Inner Primers:

| | |
|---|---|
| F: | 5′- GCAAACTTGATGGCAAA -3′ |
| R: | 5′- aagtttccaccttggagtag -3′ |

### Exon 48

### Outer Primers:

| | |
|---|---|
| F: | 5′- tgctgctaaaataacacaaatcagt -3′ |
| R: | 5′- gtccctgtgcctattgtggt -3′ |

### Inner Primers:

| | |
|---|---|
| F: | 5′- AAGCTTGAAGACCTTGAAGAGC -3′ |
| R: | 5′- cctgaataaagtcttccttaccacac -3′ |

The nPCR method involves performing an initial round of PCR using primers which form a large amplicon that encompasses the smaller amplicon formed by the primers used for the latent round of PCR and HRM. The PCR product of the first round is then used as the template for the second round of PCR. This effectively increases the concentration of template DNA (large amplicon) while simultaneously decreasing the amount of nonspecific DNA fragments and primer dimers from the WGA step.

Prepare 1 master mix for each set by mixing the following in order in a sterile microfuge tube:

| | |
|---|---|
| Sterile autoclaved water | To top up to total of 76.0 μl |
| 10x PCR Buffer | 8.4 μl |
| MgCl₂ solution | As needed for optimal efficiency |
| 10 mM dNTPs mix | 1.7 μl |
| Forward Primer 10 μM | 2.5 μl |
| Reverse Primer 10 μM | 2.5 μl |
| Polymerase (5 U/μl) | 0.8 μl |
| Total | 76.0 μl |

Deposit 1 μl of requisite DNA template into PCR plate well or microfuge tube and add 9 μl aliquots of the master mix to the template. Repeat this for all samples in the same set.
Pulse spin the PCR plate or tubes and PCR performed on the sample using the following cycling conditions:

| | | Number of |
|---|---|---|
| Temperature (° C.) | Time (s) | cycles |
| 94 | 120 | 1 |
| 94 | 30 | 35 |
| Optimal Annealing Temperature | 30 | |
| 72 | 60 | |
| 72 | 120 | 1 |
| 12 | As required | 1 |

*Note that the cycling conditions may change depending on the requirements of the polymerase and thermal cycler being used

Alternatively, the following Symmetrical Touchdown PCR protocol can also be used for all the primers specified in this disclosure:

| Temperature (° C.) | Time (s) | Number of Cycles | Notes |
|---|---|---|---|
| 95 | 120 | 1 | |
| 95 | 30 | 2 | 2° C. decrease of |
| 68-58 | 30 | (x6 annealing | annealing |
| 72 | 30 | temperatures) | temperature every |
| | | | 2 cycles |
| 95 | 30 | 35 | |
| 56 | 30 | | |
| 72 | 30 | | |
| 72 | 60 | 1 | |
| 12 | As required | 1 | |

*Note that the cycling conditions may change depending on the requirements of the polymerase and thermal cycler being used
**When using LC Green+ as the fluorescent dye, it is necessary to raise the annealing temperature by around 6° C. as the dye increases the stability of double-stranded DNA. For best results it may be necessary to re-optimise the PCR conditions in the presence of the dye.
*** For use with the outer set of primers, all the extension times at 72° C. can be increased to 40 seconds or more.

1 μl of the PCR products will be used as template for the HRM PCR. Alternatively, PCR products can be quantitated spectrometrically and diluted to suitable working concentrations. However, measured quantities may not be accurate due to difficulties in establishing a calibrating blank and the presence of various proteins.

### Protocol for Symmetrical PCR Followed by HRM (Glass Capillaries)

This protocol is for the amplification of multiple 10 μl Polymerase Chain Reaction (PCR) in LightCycler-format glass capillaries for High Resolution Melting (HRM) simultaneous mutation scanning and Single Nucleotide Polymorphism (SNP) genotyping of DMD gene amplicons. DNA template for use should be of as high purity as possible and at a quantity of at least 10 ng per reaction.

DNA probes blocked with phosphate groups at the 3′ end to prevent PCR extension are used in excess of the primers to induce the formation of probe-amplicon duplexes during and after asymmetric PCR. The probe can be designed to be complementary to either strand of the amplicon but the primer which primes the complementary strand must be present in 10-20 times higher amount than its partner. For best effect the SNIP base should be located near the middle of the probe.

List of saturating fluorescent HRM dyes and their concentrations used are given below:

| | | | 1x Final |
|---|---|---|---|
| | | | Concentration for |
| Dye | Source | 10x Concentration | HRM |
| LC | Idaho Technology | Proprietary | Proprietary |
| Green+ | | information | information |
| EvaGreen | Biotium Inc. | 20 μM | 2 μM |
| BEBO | TATAA Biocentre | 50 μM | 5 μM |
| SYTO 9 | Invitrogen | 15 μM | 1.5 μM |
| ResoLight | Roche | Proprietary | Proprietary |
| | | information | information |

*Optimal concentrations for EvaGreen, BEBO, and SYTO 9 were empirically determined while LC Green+ and ResoLight are sold in fixed concentrations

Most polymerases can be used for HRM PCR, but reaction inhibition by the HRM dye may occur. Platinum Taq (Invitrogen) has been tested to work with all 5 dyes while Amplitaq Gold (Applied Biosystems) works with all dyes except LC Green+.

HRM-capable platforms currently on the market which use LightCycler-format glass capillaries are the LightScanner32 (Idaho Technology) and Rotor-Gene Q (Qiagen), but this protocol can be applied to fit any capillary-based HRM platform.

This protocol utilizes batches of 8 individual DNA samples (henceforth referred to as a ‘set’) per pair of primers. Each set serves as 1′HRM analyses unit. LightScanner32 can accommodate 32 capillaries (4 sets) and Rotor-Gene Q can accommodate either 72 (9 sets) or 100 (12 sets) capillaries per run depending on configuration. In other words, the LightScanner32 can screen 4 primer pairs of 8 DNA samples each per run. Primer pairs to be screened together in one run must possess identical PCR amplification and HRM conditions.

Number of DNA samples screened can also be increased in multiples of 8 by correspondingly reducing the number of sets screened.

Prepare 1 master mix for each set by mixing the following in order in a sterile microfuge tube:

| | |
|---|---|
| Sterile autoclaved water | To top up to total of 76.0 μl |
| 10x PCR Buffer | 8.4 μl |
| MgCl₂ solution | As needed for optimal efficiency |
| 10 mM dNTPs mix | 1.7 μl |
| Forward Primer 0.5 μM-1 μM | 2.5 μl |
| Reverse Primer 10 μM | 2.5 μl |
| Probe 10 μM | 3.4 μl |
| 10x Fluorescent HRM dye | 8.4 μl |
| Polymerase (5 U/μl) | 0.8 μl |
| Total | 76.0 μl |

Deposit 1 μl of requisite DNA template into a glass capillary and add 9 μl aliquots of the master mix to the template. Repeat this for all samples in the same set.
8 μl of molecular biology-grade mineral oil added to the capillaries before sealing them with the capillary caps.
PCR performed on the sample using the following cycling conditions:

| | | Number of |
|---|---|---|
| Temperature (° C.) | Time (s) | cycles |
| 94 | 90 | 1 |
| 94 | 10 | 60 |
| Optimal Annealing Temperature | 10 | |
| 72 | 20 | |
| 72 | 30 | 1 |
| 12 | As required | 1 |

*Note that the cycling conditions may change depending on the requirements of the polymerase and thermal cycler being used
**When using LC Green+ as the fluorescent dye, it is necessary to raise the annealing temperature by around 6° C. as the dye increases the stability of double-stranded DNA. For best results it may be necessary to re-optimise the PCR conditions in the presence of the dye

Transfer the PCR products to a HRM-capable platform if necessary and perform an initial denaturation at 95° C. for 45 s. Cool the PCR products down to 40-45° C. to allow renaturation before performing HRM from 50° C. to 95° C. This melt will contain both the probe melt and full length amplicon melt data.
A 2^{nd} melt of the temperature region of the probes is then conducted to obtain only the probe melt peaks data. PCR products can be stored refrigerated or frozen and remelted again when necessary.
Alternatively, the following Symmetrical Touchdown PCR protocol can also be used for all the primers specified in this disclosure:

| Temperature (° C.) | Time (s) | Number of Cycles | Notes |
|---|---|---|---|
| 95 | 120 | 1 | |
| 95 | 30 | 2 | 2° C. decrease of |
| 68-58 | 30 | (x6 annealing | annealing |
| 72 | 30 | temperatures) | temperature every |
| | | | 2 cycles |
| 95 | 30 | 35 | |
| 56 | 30 | | |
| 72 | 30 | | |
| 72 | 60 | 1 | |
| 12 | As required | 1 | |

*Note that the cycling conditions may change depending on the requirements of the polymerase and thermal cycler being used
**When using LC Green+ as the fluorescent dye, it is necessary to raise the annealing temperature by around 6° C. as the dye increases the stability of double-stranded DNA. For best results it may be necessary to re-optimise the PCR conditions in the presence of the dye.
*** For use with the outer set of primers, all the extension times at 72° C. can be increased to 40 seconds or more.

### Protocol for Symmetrical PCR Followed by HRM (Glass Capillaries)

This protocol is for the amplification of multiple 10 μl Polymerase Chain Reaction (PCR) in LightCycler-format glass capillaries for High Resolution Melting (HRM) mutation scanning of DMD gene amplicons. DNA template for use should be of as high purity as possible and at a quantity of at least 10 ng per reaction.

List of saturating fluorescent HRM dyes and their concentrations used are given below:

| | | | 1x Final |
|---|---|---|---|
| | | | Concentration for |
| Dye | Source | 10x Concentration | HRM |
| LC | Idaho Technology | Proprietary | Proprietary |
| Green+ | | information | information |
| EvaGreen | Biotium Inc. | 20 μM | 2 μM |
| BEBO | TATAA Biocentre | 50 μM | 5 μM |
| SYTO 9 | Invitrogen | 15 μM | 1.5 μM |
| ResoLight | Roche | Proprietary | Proprietary |
| | | information | information |

*Optimal concentrations for EvaGreen, BEBO, and SYTO 9 were empirically determined while LC Green+ and ResoLight are sold in fixed concentrations

Most polymerases can be used for HRM PCR, but reaction inhibition by the HRM dye may occur. Platinum Taq (Invitrogen) has been tested to work with all 5 dyes while Amplitaq Gold (Applied Biosystems) works with all dyes except LC Green+.

HRM-capable platforms currently on the market which use LightCycler-format glass capillaries are the LightScanner32 (Idaho Technology) and Rotor-Gene Q (Qiagen), but this protocol can be applied to fit any capillary-based HRM platform.

This protocol utilizes batches of 8 individual DNA samples (henceforth referred to as a ‘set’) per pair of primers. Each set serves as 1′HRM analyses unit. LightScanner32 can accommodate 32 capillaries (4 sets) and Rotor-Gene Q can accommodate either 72 (9 sets) or 100 (12 sets) capillaries per run depending on configuration. In other words, the LightScanner32 can screen 4 primer pairs of 8 DNA samples each per run. Primer pairs to be screened together in one run must possess identical PCR amplification and HRM conditions.

Number of DNA samples screened can also be increased in multiples of 8 by correspondingly reducing the number of sets screened.

Prepare 1 master mix for each set by mixing the following in order in a sterile microfuge tube:

| | |
|---|---|
| Sterile autoclaved water | To top up to total of 76.0 μl |
| 10x PCR Buffer | 8.4 μl |
| MgCl₂ solution | As needed for optimal efficiency |
| 10 mM dNTPs mix | 1.7 μl |
| Forward Primer 10 μM | 2.5 μl |
| Reverse Primer 10 μM | 2.5 μl |
| 10x Fluorescent HRM dye | 84 μl |
| Polymerase (5 U/μl) | 0.8 μl |
| Total | 76.0 μl |

Deposit 1 μl of requisite DNA template into a glass capillary and add 9 μl aliquots of the master mix to the template. Repeat this for all samples in the same set.
8 μl of molecular biology-grade mineral oil added to the capillaries before sealing them with the capillary caps.
PCR performed on the sample using the following cycling conditions:

| | | Number of |
|---|---|---|
| Temperature (° C.) | Time (s) | cycles |
| 94 | 90 | 1 |
| 94 | 10 | 35 |
| Optimal Annealing Temperature | 10 | |
| 72 | 20 | |
| 72 | 30 | 1 |
| 12 | As required | 1 |

*Note that the cycling conditions may change depending on the requirements of the polymerase and thermal cycler being used
**When using LC Green+ as the fluorescent dye, it is necessary to raise the annealing temperature by around 6° C. as the dye increases the stability of double-stranded DNA. For best results it may be necessary to re-optimise the PCR conditions in the presence of the dye

Alternatively, the following Symmetrical Touchdown PCR protocol can also be used for all the primers specified in this disclosure:

| Temperature (° C.) | Time (s) | Number of Cycles | Notes |
|---|---|---|---|
| 95 | 120 | 1 | |
| 95 | 30 | 2 | 2° C. decrease of |
| 68-58 | 30 | (x6 annealing | annealing |
| 72 | 30 | temperatures) | temperature every |
| | | | 2 cycles |
| 95 | 30 | 35 | |
| 56 | 30 | | |
| 72 | 30 | | |
| 72 | 60 | 1 | |
| 12 | As required | 1 | |

*Note that the cycling conditions may change depending on the requirements of the polymerase and thermal cycler being used
**When using LC Green+ as the fluorescent dye, it is necessary to raise the annealing temperature by around 6° C. as the dye increases the stability of double-stranded DNA. For best results it may be necessary to re-optimise the PCR conditions in the presence of the dye.
*** For use with the outer set of primers, all the extension times at 72° C. can be increased to 40 seconds or more.

Transfer the PCR products to a HRM-capable platform if necessary and perform an initial denaturation at 95° C. for 45 s. Cool the PCR products down to 40-50° C. to allow renaturation before performing HRM from 65° C. to 95° C. PCR products can be stored refrigerated or frozen and remelted when necessary.

### Protocol for Asymmetrical PCR Followed by HRM (PCR Plate)

This protocol is for the amplification of multiple 10 μl Polymerase Chain Reaction (PCR) in a 96- (or 384-) well PCR plate for High Resolution Melting (HRM) simultaneous mutation scanning and Single Nucleotide Polymorphism (SNP) genotyping of DMD gene amplicons. DNA template for use should be of as high purity as possible and at a quantity of at least 10 ng per reaction.

DNA probes blocked with phosphate groups at the 3′ end to prevent PCR extension are used in excess of the primers to induce the formation of probe-amplicon duplexes during and after asymmetric PCR. The probe can be designed to be complementary to either strand of the amplicon but the primer which primes the complementary strand must be present in 10-20 times higher amount than its partner. For best effect the SNP base should be located near the middle of the probe.

List of saturating fluorescent HRM dyes and their concentrations used are given below:

| | | | 1x Final |
|---|---|---|---|
| | | | Concentration for |
| Dye | Source | 10x Concentration | HRM |
| LC | Idaho Technology | Proprietary | Proprietary |
| Green+ | | information | information |
| EvaGreen | Biotium Inc. | 20 μM | 2 μM |
| BEBO | TATAA Biocentre | 50 μM | 5 μM |
| SYTO 9 | Invitrogen | 15 μM | 1.5 μM |
| ResoLight | Roche | Proprietary | Proprietary |
| | | information | information |

*Optimal concentrations for EvaGreen, BEBO, and SYTO 9 were empirically determined while LC Green+ and ResoLight are sold in fixed concentrations

Most polymerases can be used for HRM PCR, but reaction inhibition by the HRM dye may occur. Platinum Taq (Invitrogen) has been tested to work with all 5 dyes while Amplitaq Gold (Applied Biosystems) works with all dyes except LC Green+.

HRM platforms capable of probe melt peak analyses currently on the market which use 96- or 384-well plate formats are the LightScanner (Idaho Technology) and LightCycler 480 (Roche), CFX96/384 (Biorad), or any others known to those skilled in the art. This protocol can alternatively be applied to fit any PCR plate-based or slide based or chip based HRM platform.

Roche and Bio-Rad manufacture white-welled plates optimized for use in their own platform, and it is recommended that their respective, recommended plates be used. For the LightScanner, Bio-Rad's hard-skirted white plates (Catalogue code: HSP 9655) is recommended for 96-well format, though other compatible plates can also be utilized.

This protocol utilizes batches of 8 individual DNA samples (henceforth referred to as a ‘set’) per pair of primers. Each set serves as 1′HRM analyses unit. 96-well plates can accommodate 12 sets and 384-well plates can accommodate 48 sets. In other words, 96-well plates can be used to screen 12 primer pairs of 8 DNA samples each per run. Primer pairs to be screened together in one run must possess identical PCR amplification and HRM conditions.

Number of DNA samples screened can also be increased in multiples of 8 by correspondingly reducing the number of sets screened.

Prepare 1 master mix for each set by mixing the following in order in a sterile microfuge tube:

| | |
|---|---|
| Sterile autoclaved water | To top up to total of 76.0 μl |
| 10x PCR Buffer | 8.4 μl |
| MgCl₂ solution | As needed for optimal efficiency |
| 10 mM dNTPs mix | 1.7 μl |
| Forward Primer 0.5 μM-1 μM | 2.5 μl |
| Reverse Primer 10 μM | 2.5 μl |
| Probe 10 μM | 3.4 μl |
| 10x Fluorescent HRM dye | 8.4 μl |
| Polymerase (5 U/μl) | 0.8 μl |
| Total | 76.0 μl |

Deposit 1 μl of requisite DNA template into PCR plate well and add 9 μl aliquots of the master mix to the template. Repeat this for all samples in the same set.
12 μl of molecular biology-grade mineral oil added to the wells before the PCR plate is sealed with optically-clear adhesive seal.
Subject the plate to centrifugation of 2000-3000 rpm for 60-120 seconds to separate the oil and aqueous phases.
PCR performed on the sample using the following cycling conditions:

| | | Number of |
|---|---|---|
| Temperature (° C.) | Time (s) | cycles |
| 94 | 120 | 1 |
| 94 | 30 | 60 |
| Optimal Annealing Temperature | 30 | |
| 72 | 30 | |
| 72 | 60 | 1 |
| 12 | As required | 1 |

*Note that the cycling conditions may change depending on the requirements of the polymerase and thermal cycler being used
**When using LC Green+ as the fluorescent dye, it is necessary to raise the annealing temperature by around 6° C. as the dye increases the stability of double-stranded DNA. For best results it may be necessary to re-optimise the PCR conditions in the presence of the dye

Alternatively, the following Asymmetrical Touchdown PCR protocol can be used for the asymmetric PCR probe method:

| Temperature (° C.) | Time (s) | Number of Cycles | Notes |
|---|---|---|---|
| 95 | 120 | 1 | |
| 95 | 30 | 3 | 2° C. decrease of |
| 68-58 | 30 | (x6 annealing | annealing |
| 72 | 30 | temperatures) | temperature every |
| | | | 3 cycles |
| 95 | 30 | 50 | |
| 56 | 30 | | |
| 72 | 30 | | |
| 72 | 60 | 1 | |
| 12 | As required | 1 | |

*Note that the cycling conditions may change depending on the requirements of the polymerase and thermal cycler being used
**When using LC Green+ as the fluorescent dye, it is necessary to raise the annealing temperature by around 6° C. as the dye increases the stability of double-stranded DNA. For best results it may be necessary to re-optimise the PCR conditions in the presence of the dye

Transfer the PCR products to a HRM-capable platform if necessary and perform an initial denaturation at 95° C. for 45 s. Cool the PCR products down to 40-45° C. to allow renaturation before performing HRM from 50° C. to 95° C. This melt will contain both the probe melt and full length amplicon melt data.

A 2^{nd}, melt of the temperature region of the probes is then conducted to obtain only the probe melt peaks data. PCR products can be stored refrigerated or frozen and remelted when necessary.

### Protocol for Symmetrical PCR Followed by HRM (PCR Plate)

This protocol is for the amplification of multiple 10 μl Polymerase Chain Reaction (PCR) in a 96- (or 384-) well PCR plate for High Resolution Melting (HRM) mutation scanning of DMD gene amplicons. DNA template for use should be of as high purity as possible and at a quantity of at least 10 ng per reaction.

List of saturating fluorescent HRM dyes and their concentrations used are given below:

| | | | 1x Final |
|---|---|---|---|
| | | | Concentration for |
| Dye | Source | 10x Concentration | HRM |
| LC | Idaho Technology | Proprietary | Proprietary |
| Green+ | | information | information |
| EvaGreen | Biotium Inc. | 20 μM | 2 μM |
| BEBO | TATAA Biocentre | 50 μM | 5 μM |
| SYTO 9 | Invitrogen | 15 μM | 1.5 μM |
| ResoLight | Roche | Proprietary | Proprietary |
| | | information | information |

*Optimal concentrations for EvaGreen, BEBO, and SYTO 9 were empirically determined while LC Green+ and ResoLight are sold in fixed concentrations

Most polymerases can be used for HRM PCR, but reaction inhibition by the HRM dye may occur. Platinum Tag (Invitrogen) has been tested to work with all 5 dyes while Amplitaq Gold (Applied Biosystems) works with all dyes except LC Green+.

HRM-capable platforms currently on the market which use 96- or 384-well plate formats are the LightScanner (Idaho Technology), LightCycler 480 (Roche), and CFX96/CFX384 (Bio-Rad), but this protocol can be applied to fit any PCR plate-based HRM platform.

Roche and Bio-Rad manufacture white-welled plates optimized for use in their own platform, and it is recommended that their respective, recommended plates be used. For the LightScanner, Bio-Rad's hard-skirted white plates (Catalogue code: HSP 9655) is recommended for 96-well format, though other compatible plates can also be utilized.

This protocol utilizes batches of 8 individual DNA samples (henceforth referred to as a ‘set’) per pair of primers. Each set serves as 1′HRM analyses unit. 96-well plates can accommodate 12 sets and 384-well plates can accommodate 48 sets. In other words, 96-well plates can be used to screen 12 primer pairs of 8 DNA samples each per run. Primer pairs to be screened together in one run must possess, identical PCR amplification and HRM conditions.

Number of DNA samples screened can also be increased in multiples of 8 by correspondingly reducing the number of sets screened.

Prepare 1 master mix for each set by mixing the following in order in a sterile microfuge tube:

| | |
|---|---|
| Sterile autoclaved water | To top up to total of 76.0 μl |
| 10x PCR Buffer | 8.4 μl |
| MgCl₂ solution | As needed for optimal efficiency |
| 10 mM dNTPs mix | 1.7 μl |
| Forward Primer 10 μM | 2.5 μl |
| Reverse Primer 10 μM | 2.5 μl |
| 10x Fluorescent HRM dye | 84 μl |
| Polymerase (5 U/μl) | 0.8 μl |
| Total | 76.0 μl |

Deposit 1 μl of requisite DNA template into PCR plate well and add 9 μl aliquots of the master mix to the template. Repeat this for all samples in the same set.
12 μl of molecular biology-grade mineral oil added to the wells before the PCR plate is sealed with optically-clear adhesive seal.
Subject the plate to centrifugation of 2000-3000 rpm for 60-120 seconds to separate the oil and aqueous phases.
PCR performed on the sample using the following cycling conditions:

| | | Number of |
|---|---|---|
| Temperature (° C.) | Time (s) | cycles |
| 94 | 120 | 1 |
| 94 | 30 | 35 |
| Optimal Annealing Temperature | 30 | |
| 72 | 30 | |
| 72 | 60 | 1 |
| 12 | As required | 1 |

*Note that the cycling conditions may change depending on the requirements of the polymerase and thermal cycler being used
**When using LC Green+ as the fluorescent dye, it is necessary to raise the annealing temperature by around 6° C. as the dye increases the stability of double-stranded DNA. For best results it may be necessary to re-optimise the PCR conditions in the presence of the dye

Alternatively, the following Symmetrical Touchdown PCR protocol can also be used for all the primers specified in this disclosure:

| Temperature (° C.) | Time (s) | Number of Cycles | Notes |
|---|---|---|---|
| 95 | 120 | 1 | |
| 95 | 30 | 2 | 2° C. decrease of |
| 68-58 | 30 | (x6 annealing | annealing |
| 72 | 30 | temperatures) | temperature every |
| | | | 2 cycles |
| 95 | 30 | 35 | |
| 56 | 30 | | |
| 72 | 30 | | |
| 72 | 60 | 1 | |
| 12 | As required | 1 | |

*Note that the cycling conditions may change depending on the requirements of the polymerase and thermal cycler being used
**When using LC Green+ as the fluorescent dye, it is necessary to raise the annealing temperature by around 6° C. as the dye increases the stability of double-stranded DNA. For best results it may be necessary to re-optimise the PCR conditions in the presence of the dye.
*** For use with the outer set of primers, all the extension times at 72° C. can be increased to 40 seconds or more.

Transfer the PCR products to a HRM-capable platform if necessary and perform an initial denaturation at 95° C. for 45 s. Cool the PCR products down to 40-50° C. to allow renaturation before performing HRM from 65° C. to 95° C. PCR products can be stored refrigerated or frozen and remelted when necessary.

### Primer Design

The primers are listed in Table 1.

| | | Primer | | Primer |
|---|---|---|---|---|
| | | size | | size |
| Exon | F Primer (5′-3′) | (bp) | R Primer (5′-3′) | (bp) |
| 1P | GCCAAGCACAGATA | 20 | GTAGATTGAGCCGGTGA | 19 |
| | AAGCAG | | GG | |
| 1C | CATGATTCTTTCAGT | 27 | AAGAGATTTGCAGATCCA | 23 |
| | CATTTATTATGC | | CTGTT | |
| 1L | AAAACGGATTTTTA | 25 | ATGAATTATATTTAAAGT | 30 |
| | AGATACACAGG | | TGCTTCCTAACT | |
| 1Ma | tccactgtgctattctggtttg | 22 | gtagaggcccccggatatt | 19 |
| 1Mb | agagagaaggcgggtcactt | 20 | AAAGTAACACTTCAGTTT | 28 |
| | | | TTCCTATTCG | |
| 1Mc | GGCAATTACCTTCGG | 20 | CTACTTCTTCCCACCAAA | 29 |
| | AGAAA | | GCA | |
| 1Md | CGCTGCCTTGATATA | 23 | tgcttctttgcaaactactgtgat | 24 |
| | CACTTTTC | | | |
| 2 | catcataatggaaagttactttg | 23 | agatcttaaaagtaaagtaacaaacc | 26 |
| 3a | tttttcatccgtcatcttcg | 20 | CCTCCCATCCTGTAGGTC | 20 |
| | | | AC | |
| 3b | ggaagtgtgctttgttaaattgag | 24 | cagtttctggtctgaaattctactaagtt | 29 |
| 4a | atgcctcacaggctctgttc | 20 | GCCTTGTTGACATTGTTC | 21 |
| | | | AGG | |
| 4b | gtagattgtcggtctctctgctg | 23 | acagcatccagaccttgtcc | 20 |
| 5 | ttattgcaactaggcatttggtc | 23 | cccaaaaggaaaccattcatc | 21 |
| 6a | tctatttatcactgaagatcaagga | 27 | TTTTCACTGTTGGTTTGTT | 21 |
| | ca | | GC | |
| 6b | ttgcaacaaaccaacagtga | 20 | tcagagtctaaatcaccacttttaca | 26 |
| 6c | gtttgcatggttcttgctca | 20 | GGACCCAGCTCAGGAGA | 19 |
| | | | AT | |
| 7a | aggactatgggcattggttg | 20 | TGGCAAACCACACTATTC | 20 |
| | | | CA | |
| 7b | tttgtctttgtgtatgtgtgtatgtgt | 27 | gcagtggtagtccagaaatttacc | 24 |
| 8a | tcgtcttcctttaactttgatttgt | 25 | TGGCTTCAATGCTCACTT | 20 |
| | | | GT | |
| 8b | cagATGTTGATACCAC | 24 | TGCATTTGATGATGTAGC | 22 |
| | CTATCCAG | | TGAA | |
| 8c | TGCCAAGGCCACCT | 19 | tgtgcacgtaatacctaaaaatgc | 24 |
| | AAAGT | | | |
| 9a | ccttcattctgggagtataccaa | 23 | GTGCTAGACTGACCGTGA | 22 |
| | | | Tctg | |
| 9b | tctctgcagATCACGGTC | 20 | ttatcttggaagcagttctctgg | 23 |
| | AG | | | |
| 9c | tatggtttttccccctcctc | 20 | GAATCGAGGCTTAGGGG | 20 |
| | | | AAG | |
| 10a | tgtactggaacaatctgcaaaga | 23 | AAATCTCTCCGTGTGCTT | 20 |
| | | | GC | |
| 10b | AGCACACGGAGAGA | 23 | gttggaatcccaagcacatc | 20 |
| | TTTCTAATG | | | |
| 10c | TGGACCgTTATCAAA | 21 | CTGGTCTTTCACCACTTC | 21 |
| | CAGCTT | | CAC | |
| 11a | aatctaaatgggccacaagttt | 22 | TTTTCCTGTTCCAATCAG | 21 |
| | | | CTT | |
| 11b | TGATTGGAACAGGA | 24 | ttaaccatcaaaccacatcaaaa | 23 |
| | AAATTATCAG | | | |
| 11c | GGCCGGGTTGGTAA | 20 | CATTCCCATCTTGAATTT | 24 |
| | TATTCT | | AGGAGA | |
| 12a | aatagatgcccccaaatgc | 19 | TGTTCTTTCTTCTGTTTTT | 25 |
| | | | GTTaGC | |
| 12b | TTAATGGATCTCCAG | 25 | ctagtagaaagcacgcaacataaga | 25 |
| | AATCAGAAAC | | | |
| 13a | tcaccatttgagagcaaatca | 21 | TTGTTCTTCCAAAGCAGC | 20 |
| | | | AG | |
| 13b | TGATGAATCTAGTG | 23 | cagctgattatgagtgtgtgtatattg | 27 |
| | GAGATCACG | | | |
| 14/15 | ttctagcgtacataggagactgag | 25 | cacacCTGTTCTTCAGTAAG | 23 |
| a | a | | ACG | |
| 14/15 | CAAGACAtCCTtCTCA | 22 | TGTTCACTGCATCTTCTTT | 24 |
| b | AATGGC | | TTCTG | |
| 14/15 | GCCTTTTTAGTGcAT | 21 | gaataatctatgatccaagcaaaaataaa | 30 |
| c | GGCTTT | | c | |
| 16a | agcaaatacacgcaaaagcag | 21 | GCTTCCGTCTTCTGGGTC | 19 |
| | | | A | |
| 16b | CTTCTTTCAaCACTG | 27 | tgagatagtctgtagcatgataattgg | 27 |
| | AAGAATAAGTCA | | | |
| 17a | tggtgctattttgatctgaagg | 22 | CAGCCTGTgAAATctgtgaga | 21 |
| 17b | gATTTCAcAGGCTGT | 20 | TCCACAGtAATCTGCCTCT | 23 |
| | CACCA | | TCTT | |
| 17c | ctttctagcaatgtctgacctctgt | 25 | TCCCTTGTGGTCACCGTA | 20 |
| | | | GT | |
| 17d | GGGAACAGATCCTG | 21 | tgagttttctccacttcatttgc | 23 |
| | GTAAAGC | | | |
| 18a | aaagaatgataaatgtggataaat | 27 | TGAGAAGTTGCCTTCCTT | 20 |
| | tgc | | CC | |
| 18b | CTCGCTCAGAAGCT | 20 | caaagcacggagtttacaagc | 21 |
| | GTGTTG | | | |
| 19a | ttaaggcttgaaagggcaag | 20 | GAgCTGATCTGCTGGCAT | 19 |
| | | | C | |
| 19b | CGAGAAAAAGCTGA | 22 | tgtgtttatcaaatccctaagaaga | 25 |
| | GAAGTTCA | | | |
| 20a | ttttataattaggatgtgttggctttc | 27 | ACTGGCAGAATTCGATCC | 20 |
| | | | AC | |
| 20b | GCCTCAGAACAACT | 21 | GGTGGTGGGTTGGATTTT | 19 |
| | GAACAGC | | C | |
| 20c | TCAGAAcAACATCAT | 22 | attatgctccaaatggaaggag | 22 |
| | CGCTTTC | | | |
| 21a | ggctggtgatagaggcttgt | 20 | GAAGAtCTGATAGCCGGT | 21 |
| | | | TGA | |
| 21b | TCAACCGGCTATCA | 21 | TCTTTCTCTCTGaCCTGCA | 21 |
| | GaTCTTC | | CA | |
| 21c | catactctatggcacagGATG | 23 | CATGGGTCCTTGTCCTTT | 20 |
| | AA | | CT | |
| 21d | ACTTTGTGGCCTTTA | 22 | tgtcaagttagccattttaggc | 22 |
| | CAAATCA | | | |
| 22a | taatataattgagtttgctgacaatt | 30 | CCATGTCCTGATGGCACT | 19 |
| | tagg | | C | |
| 22b | ATGCGCTATCAGGA | 20 | gcttgataagcgtgctttattg | 22 |
| | GACCAT | | | |
| 23a | gaagatcatctactttgtttacatgt | 29 | GcGCTTTCTTcGACATCTC | 20 |
| | ttg | | T | |
| 23b | TGAAAGAGATGTCg | 22 | GCTTTTGaCAATGCTCAA | 20 |
| | AAGAAAGC | | CC | |
| 23c | CTGCAAgAGCAACA | 20 | TTTCGGAGTTTATTCATTT | 23 |
| | AAGTGG | | GCTC | |
| 23d | CCTCTGAAATTAGCC | 20 | aaatgagggtagaaagtaaaatcttga | 27 |
| | GGAAA | | | |
| 24 | tgttaaaagtaatcagcacaccag | 25 | tcatacaaaattattcatattaaaggcatc | 30 |
| | t | | | |
| 25a | caattttacattttctagctatgtttc | 28 | ATCTTCTGCCCACCTTCA | 20 |
| | a | | TT | |
| 25b | tagCTTTTAGTCAGTG | 29 | CATGTGATCCCACTGAGa | 24 |
| | ATATTCAGACAAT | | GTTAAG | |
| 25c | GCCAGAGTTTGCTTC | 20 | gcaagccacagtgaaagaga | 20 |
| | GAGAC | | | |
| 26a | tctgatccccatgagttattttct | 22 | AAGGCCTCCTTTCTGGCA | 20 |
| | | | TA | |
| 26b | gtttgtttgttttgtggaagGTC | 23 | TTCATCTCTTCAACTGCTT | 24 |
| | | | TCTGT | |
| 26c | GCACGAATGGATGA | 20 | acttcaagcattgttgcatttc | 22 |
| | CACAAG | | | |
| 27a | tgcattttggatgtaaagttattttc | 26 | CTGGTAGTTGaTGGTTAG | 26 |
| | | | AGTTTCAA | |
| 27b | GAAACTCTAACCAtC | 26 | tgcctgaatgaggaattcaa | 20 |
| | AACTACCAGTG | | | |
| 27c | TCAAGCTCCACCTGT | 20 | AAGTCTTGCATTTCCCAT | 21 |
| | AGCAC | | TCA | |
| 28a | cctgctacaaagtaaaggtgaaa | 24 | CCACTTGTTTGcTTTCTCC | 20 |
| | a | | A | |
| 28b | AAgCAAACAAGTGG | 23 | tcttgggttgttttctttgga | 21 |
| | CTAAATGAA | | | |
| 28c | ttagGAAGTTTGGGCA | 21 | GCCAGGAATGTTTTCAGT | 20 |
| | TGTTG | | GG | |
| 29a | tgctccttggtgtttttaatcc | 22 | TCCTCTGAATGTCgCATC | 20 |
| | | | AA | |
| 29b | TTTGATGcGACATTC | 20 | ttagattaaagagatttttcacttatcttca | 31 |
| | AGAGG | | | |
| 29c | ttgcaaatgtgtttcagTCACT | 23 | CGCCATCTGTTAGGGTCT | 20 |
| | T | | GT | |
| 30c | caaaaaggtgattgtggaagag | 22 | TTAAGTGTAAGGAaTTTT | 26 |
| | | | CAGTCTCC | |
| 30b | TCCAGGAGTCCCTCA | 20 | aatggaagctgattcccaga | 20 |
| | CAgTC | | | |
| 30c | TGCTTGAACAGAGC | 20 | CGTCCACCTTGTCTGCAA | 20 |
| | ATCCAG | | TA | |
| 31a | tggccaatttacggtcatttt | 21 | GCCTCCTTCCCCTGATTA | 20 |
| | | | TG | |
| 31b | gactaataatgctatcctcccaac | 25 | ccaacgaaaacacgttcctt | 20 |
| | a | | | |
| 32a | ctgctttattttgtttttatttttctgt | 28 | TAGACGCaGCTCAAAATT | 20 |
| | | | GG | |
| 32b | TTGAGCtGCGTCTAC | 22 | gcgtatttgccaccagaaat | 20 |
| | AAGAAAG | | | |
| 32c | CCATGAAGTTTCGAT | 23 | TGTTTCCAATGCAGGCAA | 19 |
| | TATTCCAG | | G | |
| 33a | aggggatctctatttatttctgttcat | 27 | GGATTTTCCGTCTGCTTTT | 21 |
| | | | TC | |
| 33b | GAAGTGAAGTCTGA | 20 | ttgtggtctcagcatgcac | 19 |
| | AGTGGAAATG | | | |
| 34a | ggttataacgaaatttgaattaaag | 29 | CAACTGCTGATcTCTTTGT | 23 |
| | agta | | CAAT | |
| 34b | ATTGACAAAGAgATC | 23 | ttcacgtatgttcaaaataaccttc | 25 |
| | AGCAGTTG | | | |
| 34c | CCGTAAGATGCGAA | 20 | TCCCCAGGCAACTTCAGA | 18 |
| | AGGAAA | | | |
| 35a | ccatacagaaagccgtttca | 20 | TCTCCTTCTTGCCCAAAA | 20 |
| | | | CT | |
| 35b | CAGAGGTAGGAGAG | 20 | GGAGGTGACAGCTATCC | 23 |
| | GCCTTG | | AgTTAC | |
| 35c | AAGGAGACGTTGGT | 20 | tcgtgacagagaagggtgtaaa | 22 |
| | GGAAGA | | | |
| 36a | aataccacttaaaactaatctcaat | 30 | GGTCCACATTCTGGTCAA | 21 |
| | gaaac | | AAG | |
| 36b | tgccatggtatgtctctgtacaat | 24 | tggacattacttttcatattttatttgc | 28 |
| 37a | gcatgtgcttgctctcattt | 20 | AGTCCACCTTTGGGCgTA | 19 |
| | | | T | |
| 37b | AACCaCGGTGACCAC | 18 | agaccatttagcacaagtttcca | 23 |
| | TGC | | | |
| 37c | cttgctcatggaatatagCGTT | 23 | TGGTTGAGCTCTGAGATT | 21 |
| | T | | TGG | |
| 38a | tttagGCCTCCATTCCT | 20 | tttccactcctagttcattcacac | 24 |
| | TTG | | | |
| 38b | CCTTTGAAGGAATTG | 20 | tcattcacacttttatcacaacca | 24 |
| | GAGCA | | | |
| 39a | tttaaattggatttttgtgtgtgttt | 26 | CCTCtCgCTTTCTCTCATCT | 21 |
| | | | G | |
| 39b | GAATTGTTGCAAAG | 22 | gcatacacattgaacagaaaaagtg | 25 |
| | AGGAGACA | | | |
| 40a | aaaagatgagggacgcaaatta | 22 | GTCATCCAAGCATTTCAG | 21 |
| | | | GAG | |
| 40b | CAGTACAAGAGGCA | 20 | acgttaatagaaacaagaacatcaaca | 27 |
| | GGCTGA | | | |
| 41a | cttgcaagtcggttgatgtg | 20 | GCACTGCATTCAGcTCCT | 20 |
| | | | CT | |
| 41b | AAAGAGGAgCTGAA | 21 | acatacgtgggtttgccagt | 20 |
| | TGCAGTG | | | |
| 41c | GATCGGGAATTGCA | 20 | ATCTGAGTTGGCTCCACT | 20 |
| | GAAGAA | | GC | |
| 42a | gaaatgcttttaacactttctgga | 24 | TCAAATAAGTAGAAGGC | 27 |
| | | | ACATAAGAAA | |
| 42b | GCACACTGTCCGTG | 20 | TTCAGAGACTCaTCTTGCT | 25 |
| | AAGAAA | | TAAAGA | |
| 42c | TCCTGACCTCTGTGC | 16 | tgaaagtgctttggttt | 17 |
| | T | | | |
| 43a | ccatttgctcctttgggatt | 21 | CACAGGCGTTGCACTTTG | 18 |
| 43b | GCAAGAAGACAGCA | 20 | tgaaaacaaatcatttctgcaag | 23 |
| | GCATTG | | | |
| 44a | tgcaaatgcaggaaactatca | 21 | AAACgCCGCCATTTCTC | 17 |
| 44b | ATGGCGGcGTTTTCA | 17 | aagagtccagatgtgctgaaga | 22 |
| | TT | | | |
| 44c | tgcagGCGATTTGACA | 18 | TTCAGCTTCTGTTAGCCA | 22 |
| | GA | | CTGA | |
| 45a | ttcacatggagcttttgtatttct | 24 | AGTTTGCCaCTGCCCAAT | 18 |
| 45b | ATTGGGCAGtGGCAA | 18 | aaatgttttcattcctattagatctgtc | 28 |
| | ACT | | | |
| 45c | gggcttcatttttgttttgc | 20 | TTTCTTCCCCAGTTGCATT | 20 |
| | | | C | |
| 46a | ttaaattgccatgtttgtgtcc | 22 | TTGCTGCTCTTTTCCAGG | 20 |
| | | | TT | |
| 46b | ttctccaggCTAGAAGAA | 23 | cctgggggatttgagaaaata | 21 |
| | CAAAA | | | |
| 47a | tttcagtcaatcagctctgtgc | 22 | GGGCAACTCTTCCACCAG | 20 |
| | | | TA | |
| 47b | cgttgttgcatttgtctgtttc | 22 | gacggaagagatggttaatgtctaa | 26 |
| 48a | tgctaaaataacacaaatcagtaa | 29 | TTTCAAGCTGCCCAAGGT | 18 |
| | gattc | | | |
| 48b | cagGTTTCCAGAGCTT | 22 | CGTCAAATGGTCCtTCTTG | 20 |
| | TACCTG | | G | |
| 48c | CTGCTGCTGTGGTTA | 21 | tgataccaaatgagaaaattcagtg | 25 |
| | TCTCCT | | | |
| 49a | tctgtttcttttctctgcacca | 22 | CCGGTTGTTTAGCTTGAA | 21 |
| | | | CTG | |
| 49b | ctgcactatatgggttcttttcc | 23 | gcaaatgtacaacaggggaag | 21 |
| 50a | tcctttaaaagaaattctacccact | 27 | GTTTACCGCCTTCCACTC | 20 |
| | aa | | AG | |
| 50b | cgaataagtaatgtgtatgcttttct | 27 | ccaaagagaatgggatccag | 20 |
| | g | | | |
| 51a | tgaaattggctctttagcttgtg | 23 | TGAAATCTGCCAGAGCA | 21 |
| | | | GGTA | |
| 51b | TGCCATCTTCCTTGA | 20 | aaacttctgccaacttttatcatttt | 26 |
| | TGTTG | | | |
| 52a | aaaagtgttttggctggtctc | 21 | GGCAGCGGTAATGAGTTC | 20 |
| | | | TT | |
| 52b | GGCAACAATGCAGG | 19 | ttgtgtgtcccatgcttgtt | 20 |
| | ATTTG | | | |
| 53a | aaatgtgagataacgtttggaagt | 24 | TCCTTTAACATTTCgTTCA | 23 |
| | | | ACTG | |
| 53b | TTGAAtGAAATGTTA | 25 | ccagtattttattttaaaCaggtatctttg | 30 |
| | AAGGATTCAA | | | |
| 53c | TGGGATGAAGTACA | 23 | TCCTTAGCTTCCAGCCAT | 20 |
| | AGAACACCT | | TG | |
| 54a | gacctgaggattcagaagctg | 21 | GTTTCAGGGCCAAGTCAT | 20 |
| | | | TT | |
| 54b | GCCAGTGGCAGACA | 20 | tcaccaccccattattacagc | 21 |
| | AATGTA | | | |
| 55a | caactcaccccattgttggta | 21 | TTCtTCCAAAGCAGCCTCT | 20 |
| | | | C | |
| 55b | GGCTGCTTTGGAaGA | 20 | agcggaaatgcctgacttac | 20 |
| | AACTC | | | |
| 55c | tctgaacatttggtcctttgc | 21 | GCTTCTGTAAGCCAGGCA | 20 |
| | | | AG | |
| 56a | tatattttgcaattctccaaattcac | 26 | GACTGCATCATCGGAACC | 21 |
| | | | TT | |
| 56b | TCCAAGGTGAAATT | 21 | tttttgctccacatcttttcc | 21 |
| | GAAGCTC | | | |
| 57a | attcaagActgctttgtagttcaca | 25 | GCCACACCAGcAAGTTCC | 18 |
| 57b | GGAACTTgCTGGTGT | 20 | aaggcacgaggcttaaaaatg | 21 |
| | GGCTA | | | |
| 57c | GAAGCGTCTGCACC | 20 | TCTGCTTCTGAACTGCTG | 20 |
| | TTTCTC | | GA | |
| 58a | gacctgggagtttcataaacaag | 21 | GCTGCTCTGTCAGAAATA | 22 |
| | | | TTCG | |
| 58b | cacttcttttcatctcatttcacag | 25 | ccgtcaccactgatccttc | 19 |
| 59a | ccagtatgacctttttgacaatg | 23 | GGAGtGCAGGTTCAATTT | 21 |
| | | | TTC | |
| 59b | GAAAGCAGGCTGAG | 19 | CAGCTTGGtGCAGCTTGA | 18 |
| | GaGGT | | | |
| 59c | CCCTTGAAAGACTCC | 21 | ggccctgaagcaaagaagtag | 21 |
| | aGGAAC | | | |
| 60a | actggcactgcaccctaaag | 20 | AGAGCTGAATGCCCAAA | 20 |
| | | | GTG | |
| 60b | CCTTGCTCGCCAGCT | 18 | actttcattgttctttacaaattttatctg | 30 |
| | TAC | | | |
| 61 | gaaaggatacattgttttaattgttc | 27 | tgcaataaagttaagtgataaaagctg | 27 |
| | c | | | |
| 62 | tgtctttcctgtttgcgatg | 20 | aatgctcttttaaaaatgtgatacttcc | 28 |
| 63 | catgttgttgttattgttgttttcttt | 27 | catttaacttggaggaaacatgg | 23 |
| 64 | gggtgggttgtctgttatttct | 22 | ccaacctactttttattctaagcaaag | 27 |
| 65a | ttattaaagggtatgagagagtcct | 28 | AGGATATCCATGGGCTGG | 19 |
| | agc | | T | |
| 65b | GCACAACCTCAAGC | 20 | cacttcaccattctgtacgctaa | 23 |
| | AAAATG | | | |
| 66a | tttttccttttcaaggctttattc | 24 | ACACGGATCCTCCCTGTT | 19 |
| | | | C | |
| 66b | aaagggtcagtaattgttttctgc | 24 | aaaagaatacagcattagtatacacgactt | 30 |
| 67a | ctgtggaaatactggctactcttg | 24 | TGGATAGAATCATGCAG | 22 |
| | | | AAGGA | |
| 67b | GCAAGTGGCAAGTT | 20 | aacttacAAAcTGGAAGCAGC | 23 |
| | CAACAG | | TC | |
| 67c | CTTTGGGGGCAGTA | 20 | ggttccaaatatcccaaatcc | 21 |
| | ACATTG | | | |
| 68a | gctttgcaaccattgttctttc | 20 | GGGGTTCCAGTCTCATCC | 19 |
| | | | A | |
| 68b | AATAATAAGCCAGA | 23 | aaaggtgaaaaatgatgagaaaaa | 24 |
| | GATCGAAGC | | | |
| 69a | ttgtgggactaatgaacattgc | 22 | CCTTTTGCAACTCGACCA | 19 |
| | | | G | |
| 69b | ATTATGACATCTGCC | 22 | actgaaatttatcccaggtgaact | 24 |
| | AAAGCTG | | | |
| 70a | agtgtcatggggcagaagac | 20 | AGTCTGCACTGGCAGGTA | 20 |
| | | | GC | |
| 70b | AAGGTATTTTGCGA | 21 | gggtgttcagctgagaggag | 20 |
| | AGCATCC | | | |
| 71 | ggtttggctattgctttcca | 20 | cagcacccttcagcaaaaa | 19 |
| 72 | gaataaaagcattctaggccatgt | 24 | gcctggcatacaactagtctca | 22 |
| 73 | tttcaggaatgttcgattaggtc | 23 | agcaatttcattgtcaggaaca | 22 |
| 74a | tctattttcaaatacactcctgagtc | 27 | TTTCCTCACTCTCTAAGG | 26 |
| | c | | AAATCAAG | |
| 74b | AATCCAGCATTACTG | 21 | ggcacttttctatgtgtgcaag | 22 |
| | CCAAAG | | | |
| 75a | ccatggtatataaaatttggtgatg | 26 | AGAGGTGGGCATCATTTC | 20 |
| | a | | AG | |
| 75b | CATAAAGGCCTGTC | 20 | TCCAgCAGCTGCCTTAGC | 18 |
| | CCCACT | | | |
| 75c | AGGATGCAAATCCT | 20 | aagtgctctctgaggtttagttttg | 25 |
| | GGAAGA | | | |
| 76a | cagtgaaatatcccaaattaaaca | 27 | ACACCGTTGTGCCATTCA | 19 |
| | atc | | C | |
| 76b | aattctgttttcttttggatgacttag | 27 | cctttcttcagacaacaaaatctg | 24 |
| 77 | catggccctttaatatctgttttc | 24 | agcaaatctgagtcccttctagg | 23 |
| 78 | ggtaaaagaagcaaattggtatg | 20 | gccgtgagcctgaatctc | 18 |
| | aa | | | |
| 79a | tgcagcttctgtgttgtcttc | 21 | ACTAAGGACTCCATCGCT | 21 |
| | | | CTG | |
| 79b | CTTTTCCACATGGCA | 20 | GCGGGAATCAGGAGTTG | 20 |
| | GATGA | | TAA | |
| 79c | GCAGAGCGATGGAG | 20 | AATACCACTACCCTTCAC | 29 |
| | TCCTTA | | AAAAATATAGA | |
| 79d | GAGGATTAGACAGT | 29 | AAATGGCAAGTTATTTAG | 26 |
| | AAGAGTTTACAAGA | | CTATCAAG | |
| | A | | | |
| 79e | CAGGTTTTACACGTC | 23 | AAAGCACACTTTAGTTTA | 28 |
| | TATGCAAT | | CAATCTTTCT | |
| 79f | TCTTTATATGGAACG | 23 | TGGTAGAGGAAGTCTTAT | 29 |
| | CATTTTGG | | CTTTAATATGC | |
| 79g | GCATTGTTTTGCATC | 21 | ACTCAAGCCTGCCCCACT | 18 |
| | CTTTTG | | | |
| 79h | GACTTCCTCTACCAC | 21 | TTCTTTCTATTAGGATGT | 26 |
| | CACACC | | GACATGAA | |
| 79i | GCAGGCTTGAGTTTT | 21 | TCCGGGAAAAATCCATTC | 20 |
| | CATTTC | | TA | |
| 79j | CAGGCTTACCTGCTT | 19 | TCTGATATAATAAGTCCT | 30 |
| | GGTC | | GTGTATTCATTC | |
| 79k | TTTCCACTGACAACG | 23 | CCTTCTGATTGATTTCCA | 22 |
| | AAAGTAAA | | CTGA | |
| 79m | GCTAGCAATGCCAC | 21 | GTGGCCTACTCCTTCACA | 20 |
| | GATTTAG | | GG | |
| 79n | CTCCCAAGCAGTAG | 20 | CAAAAACCAAACCACCC | 20 |
| | CAGGAC | | AGT | |
| 79o | TGCCCTCTTCTCACA | 20 | GGCTTCTGGGTTGATACC | 20 |
| | GTCAA | | TG | |
| 79p | TAAGACAGTAGCCC | 21 | CTAATCCACCAAGAAGG | 22 |
| | CATCACA | | GTTTT | |
| 79q | TCCACACAGGTTTGT | 27 | AAAAGAAATAAAATGGC | 25 |
| | AAGTAAGTAAGA | | ATGAAAGA | |
| 79r | GGAGAAAAGCTCAA | 22 | CACTTGATGTCAGCCCAC | 20 |
| | GAGGAAAA | | TC | |
| 79s | TTGCAAATCTGTTAC | 23 | ACACCCCAAAACCAAAG | 20 |
| | CTCTGACA | | TGA | |
| 79t | ACATCAAACACGGC | 20 | AGAGCTTTGGGTTTTCTT | 22 |
| | TTCTCA | | TTGA | |
| 79u | CACAGCTTCACCACT | 20 | AATCCTTGGGTAAAGAA | 23 |
| | TGTCC | | AAGGTC | |
| 79v | TTGGTTTTTGTCTTG | 22 | AATTATTTATGCACTCTA | 29 |
| | CATTTTT | | TTTACCTCTGA | |
| 79w | AAAACAAGGGGTTA | 25 | TTAAAATGCAGCAATAA | 24 |
| | CTTTACATCCT | | AGCTCTC | |
| 79x | GACACATTAGCTCTG | 24 | TCAGTACCTGAAAACAAT | 25 |
| | GAGTGAGTC | | GACAAAA | |
| 79y | ACCACCGAGTATTA | 26 | TGGTGAAGTCTGATATGT | 24 |
| | AACTGTAAATCA | | TGTGAA | |
| 79z | CACCAACACTGTAA | 24 | acaaagtactgcagtccagtcaa | 23 |
| | CATTTACGAA | | | |

The probes are listed in table 2.

| | | Pro | |
|---|---|---|---|
| E | | [symbol] Size | |
| [symbol] | Probe Sequence | (bp) | SNP |
| 6 | GATTGCAACaAACCAACAGTGAAAAG | 26 | rs1800256 |
| | | | (A/C) |
| 8 | CATTTTCAGcTACATCATCAAATG | 24 | rs1800264 |
| | | | (C/T) |
| 9 | ttctctgcagATCACgGTCAGTCTAGCACA | 30 | rs1800265 |
| | | | (A/G) |
| 1 | CATTGCAAGCACAcGGaGAGATTTC | 25 | rs1800266 |
| | | | C), rs72470507 |
| 1 | TAAGCTGATTGGAaCAGGAAAATTATCAGA | 30 | rs34155804 |
| | | | (67.6% T/A) |
| 1 | ACATCTGTAGaTGGACAGAAG | 21 | rs5927083 |
| P1 | | | 9% C/97.1% T) |
| 1 | CAAGACAtCCTgCTCAAATG | 20 | rs1800257/ |
| P2 | | | T), rs1800258 |
| | | | (G/T) |
| 1 | CTTTTTAGTGcATGGCTTTCA | 21 | rs5972599 (A/ |
| P3 | | | 100% G) |
| 1 | TTCAcTcAAACAAGATCTTCTTTCAaCACT | 30 | rs1800259/ |
| | | | C), rs1800267 |
| | | | (100% C/T), |
| | | | 2468692 (T/C) |
| 1 | GAGTACAGtACAGgttagtg | 20 | rs41309715 |
| | | | (A/G) |
| 1 | cttctcacagATTTcACAGGCTGTCACCAC | 30 | rs34563188 |
| | | | (A/G) |
| 1 | CAAAAGAAGAGGCAGATTaCTGTGG | 25 | rs16998350 |
| | | | (A/66.7% T) |
| 1 | GATGCCAGCAGATCAGcTCAGGCCCTGG | 28 | rs1800260 |
| | | | (C/G) |
| 2 | TCAACCGGCTATCAGaTCTTCAAC | 24 | rs41312094 |
| | | | (A/G) |
| 2 | GATGTGCAGGtCAGAGAGAAAG | 22 | rs228406 |
| | | | 8% C/13.2% T) |
| 2 | GTGAAAGAGATGTCgAAGAAAGCGCC | 26 | rs1800268 |
| | | | (A/G) |
| 2 | TGGTTGAGCATTGtCAAAAGCTAGAGGAGC | 30 | rs1800261 |
| | | | (C/T) |
| 2 | GAACTTAACtCTCAGTGGGATC | 22 | rs3827462 |
| | | | 4.1% T, 5.9% A) |
| 2 | TACAGAAAGCAgTTGAAGAGATGAAGgta | 29 | rs1800262 |
| | | | (G/T) |
| 2 | CTTGAAACTCTAACCAtCAACTACCAGTGG | 30 | rs1800269 |
| | | | (C/T) |
| 2 | TGGAGAAAgCAAACAAGTGGCTAAATGAAG | 30 | rs1800270 |
| | | | (C/G) |
| 2 | ATTTGATGcGACATTCAGAGGATA | 24 | rs5030730 |
| | | | (C/T) |
| 3 | AGGAGACTGAAAAtTCCTTACACTTAATC | 29 | rs1800263 |
| | | | (A/T) |
| 3 | CCAGGAGTCCCTCACAgTCATTGAC | 25 | rs28715870 |
| | | | (C/100% A) |
| 3 | GCCAATTTTGAGCtGCGTCTACAAGAAAGT | 30 | rs1057872 |
| | | | (T/100% A) |
| 3 | GAATTGACAAAGAgATCAGCAGTTGA | 26 | rs36072930 |
| | | | 8.7% C/1.3% T) |
| 3 | AGTAAcTGGATAGCTGTCACCTCCCGAGCA | 30 | rs1801185 |
| | | | (C/T) |
| 3 | AGAAGAGTGGTTAAAtCTTTTGTTGgtaag | 30 | rs16990264 |
| | | | (66.7% A/T) |
| 3 | GAATGACATAcGCCCAAAGGTGGACTCTACACGTG | 37 | rs34102501 |
| | | | 4% A/98.6% G*) |
| 3 | CAAGCAGCAAACTTGATGGCAAACCaCGGTGACCA | 39 | rs1801187 |
| | C | | .1% A/23.9% G) |
| 3 | AATGAAGACAgTGAGGGTACT | 21 | rs1800271 |
| | | | (A/G) |
| 3 | GAGAAAGcGaGAGGAAAT | 18 | rs1064325 |
| | | | (A/100% C), |
| | | | 1801186 (A/C) |
| 4 | GAAAGAGGAgCTGAATGCAGTGCG | 24 | rs1800272 |
| | | | (A/G) |
| 4 | CTCTTTAAGCAAGAtGAGTCTCTGAAGgta | 30 | rs331312 |
| | | | (A/100% C) |
| 4 | TGAGAAATGGCGGcGTTTTCATTATG | 26 | rs16990169 |
| | | | (A/G) |
| 4 | CATTGGGCAGtGGCAAACTG | 20 | rs1800273 |
| | | | (T/100% C) |
| 4 | TAACCAACCAAACCAAGAgGGACCAT | 26 | rs1800274 |
| | | | (A/G) |
| 4 | TGACGTTcAGgtagggaactttttgc | 26 | rs1800275 |
| | | | .1% A/11.9% C) |
| 5 | GCAACAGTTGAAcGAAATGTTAAAGGATTC | 30 | rs1801188 |
| | | | .4% C/64.6% T) |
| 5 | CGAGAGGCTGCTTTGGAaGAAACTCAT | 27 | rs1801189 |
| | | | (A/G) |
| 5 | CAGGAACTTgCTGGTGTGGCTAC | 23 | rs35527662 (-/ |
| | | | C) |
| 5 | GGCTGAGGtGGTCaATACTGAG | 22 | rs1800278 |
| | | | .4% A/1.6% G), |
| | | | 1305353 (1.6% |
| | | | A/98.4% T) |
| 5 | TGAACCTGCaCTCCGCTGAC | 20 | rs1800279 |
| | | | 6% A/3.4% G), |
| 5 | GAAAGACTCCaGGAACTTCAAG | 22 | rs1800280 |
| | | | .3% A/8.7% G) |
| 6 | GGAGCTGCTTCCAgTTTgtaag | 22 | rs12690372 |
| | | | (C/100% T) |
| 7 | GCTAAGGCAGCTGcTGGAGCAAgtgagg | 28 | rs1800281 |
| | | | (C/T) |
| 7 | ttattaaaggGAAgAAATACCCCTG | 25 | rs1795743 |
| | | | (A/100% G) |
| 7 | GGGCAGAGCgATGGAGTCCTTAGTATC | 27 | rs16989352 |
| | | | (100% C/T) |

[symbol]

indicates data missing or illegible when filed

The Nested PCR primers are listed in Table 3:

| | | Primer | | Primer |
|---|---|---|---|---|
| | | size | | size |
| Exon | F Primer (5′-3′) | (bp) | R Primer (5′-3′) | (bp) |
| 1 | tccactgtgctattctggtttg | 22 | Tgcttctttgcaaactactgtgata | 25 |
| 2 | gcctggccatttttcaga | 18 | Atatttccagatttgcacagctaa | 24 |
| 3 | tttttcatccgtcatcttcg | 20 | Actttttctgcaggcggtag | 20 |
| 4 | ttggtttatgctaaaaacgtacca | 24 | Catgtgctctcagtaagaactgg | 23 |
| 5 | gtgattttacacatgcattttgttt | 25 | Tgtcaatttaaaaagcagcactatg | 25 |
| 6 | ggcatagataccaatgaatcagaa | 24 | Tggaaccatactggggaaaa | 20 |
| 7 | aggactatgggcattggttg | 20 | Agacaattcataatatagtgcaaga | 28 |
| | | | agg | |
| 8 | tggaggacattcatggacaa | 20 | Tcaatgaagcaaaattgaaaagg | 23 |
| 9 | aagtgccttcattctgggagt | 21 | Ggcactgaaaaattcaagcaa | 21 |
| 10 | tcctgtgtttgataatgccagt | 22 | Tcaatggttgctctccaaaa | 20 |
| 11 | gtggttttgggattctgcaa | 20 | Ccaaccagtctcccttgaaa | 20 |
| 12 | ttccggggtgactgatagtg | 20 | Tcaagccattgcaacaaaga | 20 |
| 13 | gctgagcgtcatagcagaaa | 20 | Tttacccatccgcagttagtt | 21 |
| 14 & | tcagaaagagtgtcccttcca | 21 | Tgaataatctatgatccaagcaaaa | 25 |
| 15 | | | | |
| 16 | ttctgaacttttgatcctttgc | 22 | Ctggttgcttcttttgtaggg | 21 |
| 17 | ttggcttcaatatggtgctattt | 23 | Gtacccgaggattctggaaa | 20 |
| 18 | agaggtgtcaggcaggagtc | 20 | Tcagtcacagaagaatccagaaa | 23 |
| 19 | acgtgataagctgacagagtgaa | 23 | Catcccattttcttccaatga | 21 |
| 20 | tttgaaatcattcatgtggtga | 22 | Aatccgggtatctacgtcttaca | 23 |
| 21 | ggctggtgatagaggcttgt | 20 | Ggttgtagggagaatggttcc | 21 |
| 22 | aggtccctggcatattacaca | 21 | Tgggcaaactaccatacttgtc | 22 |
| 23 | gaagatcatctactttgtttacatgttt | 29 | Taagcaaatcgccatccttt | 20 |
| | g | | | |
| 24 | tcatacagtagaccataaaaatgcag | 28 | Atctaaccaccacccccaac | 20 |
| | tc | | | |
| 25 | gcaagactgttaggcagtcatct | 23 | Ggaacaaagccttaaccaaaag | 22 |
| 26 | tctgatccccatgagttattttct | 24 | Tctcttagaaccaggaaagagca | 23 |
| 27 | tctaactgggatgttgtgagaaag | 24 | Taagcaaatggcccaaagtc | 20 |
| 28 | tgtctagctgcattttgaattacc | 24 | Tccaaagtaccagtgctgagtg | 22 |
| 29 | ttgaagcaaaaatgctccttg | 21 | Cagtgtctggcattggattg | 20 |
| 30 | ggaagctgcgaaatctgtct | 20 | Atcaaaacaaccccatggaa | 20 |
| 31 | ttacaggtgtgaaccaccactc | 21 | Tgtcctcaaatccaatcttgc | 21 |
| 32 | ttcctgtgttggatgaatgg | 20 | Gccacaatacatgtgccaat | 20 |
| 33 | accgctgcaaaatgctactc | 20 | Agagagagagaggtgtttagaattg | 26 |
| | | | c | |
| 34 | ggttataacgaaatttgaattaaaga | 29 | Cttaccattggttttatcatggtc | 24 |
| | gta | | | |
| 35 | taaaaccttgcagaatcatagtaacc | 26 | Gttatagatattgaattaagagccag | 28 |
| | | | ca | |
| 36 | ccacttaaaactaatctcaatgaaact | 30 | Gggaaagataaaggaaggaagaa | 23 |
| | tta | | | |
| 37 | gcatgtgcttgctctcattt | 20 | Ttggcattcattttccttttg | 21 |
| 38 | ccagttgtatttcaaaacccttc | 23 | Aaccgtaagtgctcctatattaccat | 26 |
| 39 | aaaagaaaggctatgagcacagt | 23 | Gcaacacatcgttcaaaatca | 21 |
| 40 | aggtttcttagcttcctatacatgg | 25 | Cacaatacaaggaaatgcatca | 22 |
| 41 | gggttattgagcgaggatga | 20 | Tgagggaaaccactcactttc | 21 |
| 42 | gaaatgcttttaacactttctgga | 24 | Gtcaattgttctggcactatgaa | 23 |
| 43 | aataattaagaattgcaacaccatttg | 27 | Gagagtgatacttctttttccctgtc | 26 |
| 44 | tttaaaatgttgtgtgtacatgctagg | 27 | Ttccatcacccttcagaacc | 20 |
| 45 | aaattttcacatggagcttttgt | 23 | Tctttaatgttagtgcctttcacc | 24 |
| 46 | tggccaggaatttttgaatc | 20 | Cctaatgggcagaaaaccaa | 20 |
| 47 | tgagggggtgagtgtttca | 19 | Atagccaaagcaaacggtca | 20 |
| 48 | tgctgctaaaataacacaaatcagt | 25 | Gtccctgtgcctattgtggt | 20 |
| 49 | tctgtttcttttctctgcacca | 22 | Aaagacagctttgcctctgc | 20 |
| 50 | tggagaaagggtttttgtatgg | 22 | Ccatatcccgttgtcatgc | 19 |
| 51 | gtcatgaataagagtttggctca | 23 | Gcctaagaactggtgggaaa | 20 |
| 52 | tttgagcctttaaatgaagaaaatc | 25 | Aaatgtgagggggatatatgaact | 24 |
| 53 | tcctgttgttcatcatcctagc | 22 | Tttcagctttaacgtgattttctg | 24 |
| 54 | ctggttattgctcaagatgctg | 22 | Gtctgagccaagtccgtga | 19 |
| 55 | gcaacaactcaccccattgt | 20 | Tcctccttgtccaaataccg | 20 |
| 56 | tcttcttgttagtttagcctgattga | 26 | Tcatctccataccaagcaaataaa | 24 |
| 57 | tcctctgttttgtggctctca | 21 | Tgacccttgggtgagaagag | 20 |
| 58 | attaacagatagacccatatagccttt | 27 | Atgcatggtagtttatgagtcctt | 24 |
| 59 | aatcagtaggttaccctcttgttca | 25 | Tgaatttgtgaaagacggactg | 22 |
| 60 | gggggacacaaacattcaaa | 20 | Taaaatatcctatcctcacaaatatta | 30 |
| | | | cca | |
| 61 | cgagtctggaatactatatacggtaa | 27 | Ccttctcaacttatcaaccaagtta | 25 |
| | g | | | |
| 62 | cagcAggttaatccatcctgt | 21 | Gtcgttccccattgcatc | 18 |
| 63 | cgaatggttcaaaagcaaaaa | 21 | Aaacttgtttttaaggaacctctgat | 26 |
| 64 | tcttttccccacgttaacaga | 21 | Gcaaactctaggccaaggatac | 22 |
| 65 | tgtggttcacgtttggtgtt | 20 | Ttgcaaaatgagtgtctcagg | 21 |
| 66 | ggaaaagttcaaaacaatcttaataa | 29 | Cacaaaccaaattttatgacactcta | 27 |
| | aac | | a | |
| 67 | ccccactctgtggaaatactg | 21 | Ggttccaaatatcccaaatcc | 21 |
| 68 | cagcctagctttgcaacca | 19 | Cagcccaatcaccttctctc | 20 |
| 69 | gagaggcaccggaaaaatct | 20 | Tttagctggggaacatctgg | 20 |
| 70 | ttgctcacaiccacaacactc | 21 | Ggaaagtggcaactggacat | 20 |
| 71 | caactgtgtgtttttcttttaattcac | 27 | Gattctcctgatctgtgtagatgg | 24 |
| 72 | catagaattagaataaaagcattcta | 29 | Atgcacgttagagggcaagt | 20 |
| | ggc | | | |
| 73 | ggtctaccacacactgcctca | 21 | Aatgaatggctccttaaaagatca | 24 |
| 74 | caaaaggtctttagaagcaggaa | 23 | Tctgcaaatggagctaaacaga | 22 |
| 75 | gcctcttttgcttgctgttc | 20 | Tcactttgcaggcacatacc | 20 |
| 76 | cagtgaaatatcccaaattaaacaat | 27 | Ttctgtgggccagatagagc | 20 |
| | c | | | |
| 77 | ctgtatggatttcttcttcccttt | 24 | Ttttcaccatggacccaaat | 20 |
| 78 | tccagcagagaataaatggtca | 22 | Gtgatgacaacggcaatgaa | 20 |
| 79α | tcttgtgcttatctatggaattctttt | 27 | GcATAATACCACTACC | 20 |
| | | | CTTCACAA | |
| 79β | TGACAGATGAAGAAG | 22 | TGGTGTGGTGGTAGA | 20 |
| | GAGCAGA | | GGAAGT | |
| 79γ | AACTTTGAGGCAGCG | 19 | CAACCAACCGATTACT | 25 |
| | CATT | | CACTCTG | |
| 79δ | TTCCACTGACAACGAA | 23 | CCCACATGGGACAAT | 21 |
| | AGTAAAG | | AAATCT | |
| 79ε | TGCCCTCTTCTCACAG | 20 | CACTTGATGTCAGCCC | 21 |
| | TCAA | | ACTC | |
| 79ζ | GCTAAGGACTGGTAG | 23 | AAAATCCTTGGGTAA | 23 |
| | GAAAAAGC | | AGAAAAGG | |
| 79η | CATGAAGATTTGGTTT | 20 | TGGTGAAGTCTGATAT | 24 |
| | TTGTCTTG | | GTTGTGAA | |
| 79θ | TTGGACATTATTTCGT | 23 | Ttgattgatatgggggaagg | 20 |
| | GTTGTGT | | | |

### Mutation Screening of Duchenne's Muscular Dystrophy Patients

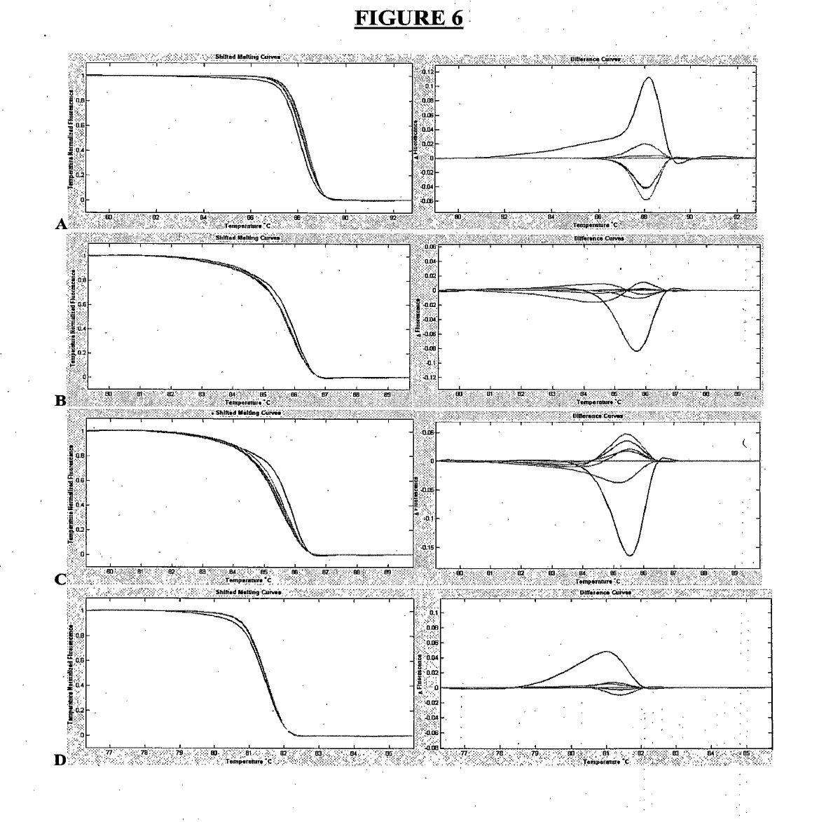

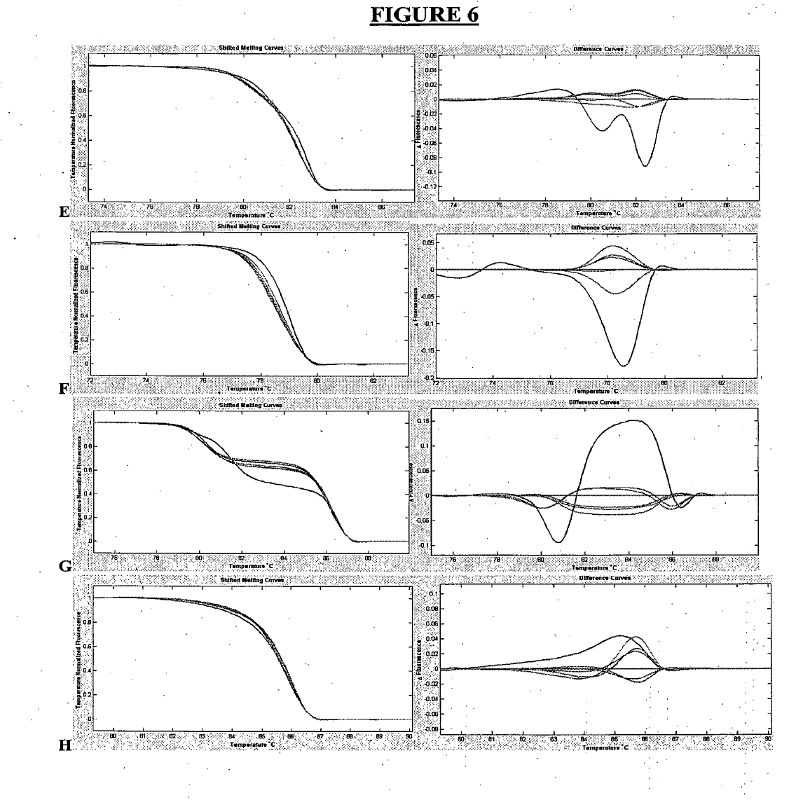

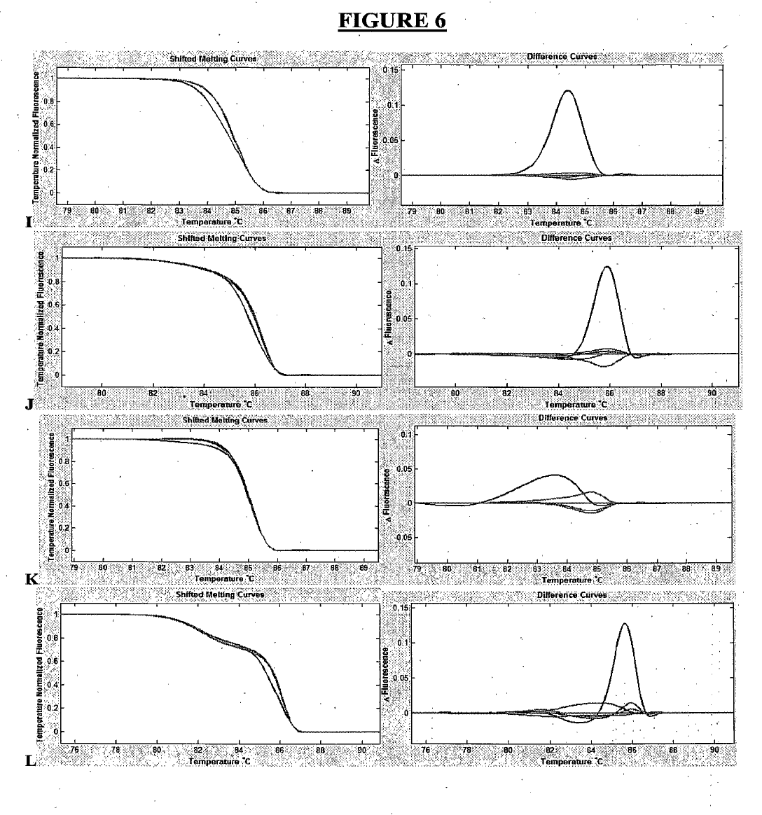

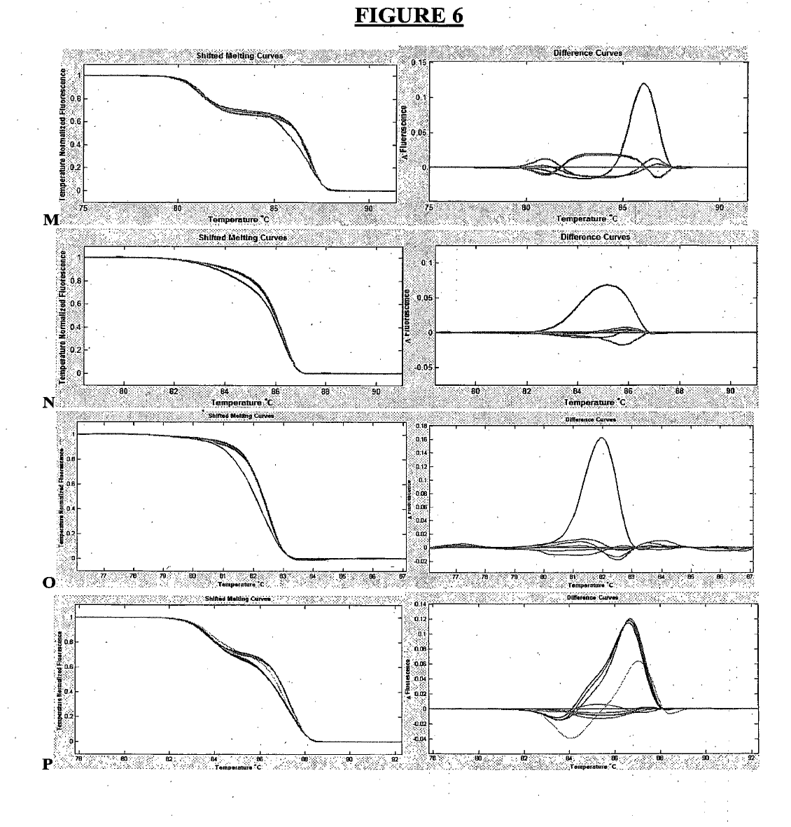

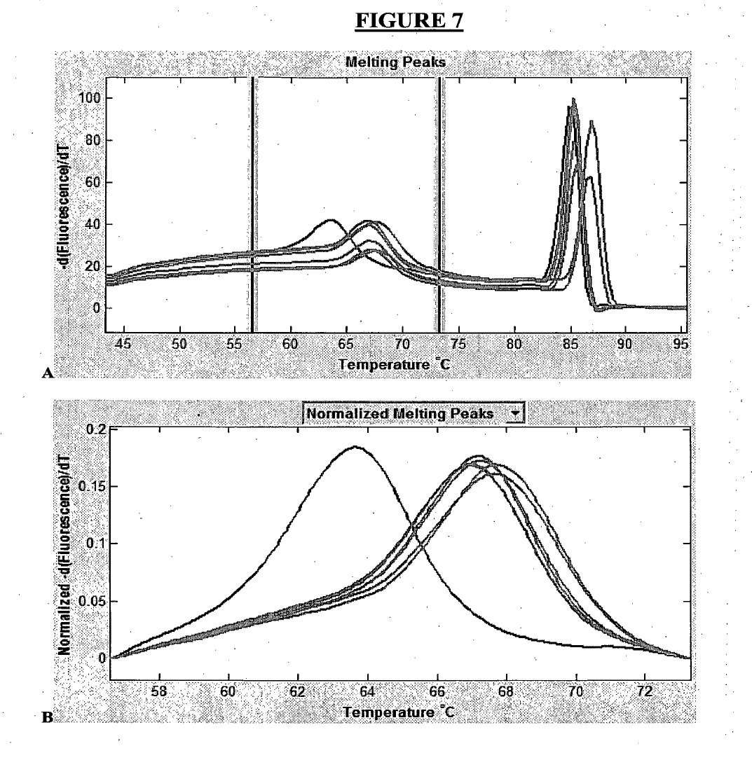

Examples of the validation of the method for mutation screening in clinical samples for Duchenne's Muscular Dystrophy (DMD) is shown in FIG. 6 and FIG. 7. The table shows the primers used for the HRM analysis of the respective patient sample carrying a known mutation as listed in the corresponding exon. DNA samples from patients were analyzed and compared against DNA samples from 6 normal individuals (Grey). Red, blue, or green melt curves indicate outliers deviating from the normal samples which act as references. The Unspiked HRM results are: given in FIG. 6A-H, and the Spiked results in FIG. 6I-N. FIG. 6O-P show Carrier samples and how they compare to Patient and Wild-Type/Normal samples. The results for Patient 414/418/432 show the probe melt peaks (FIG. 7). The mutation creates a mismatch in the patient DNA, causing the probe to melt earlier than the perfect complementary match found in normal samples. Mutation nomenclature uses the NM_{—}004006.2 version of the cDNA for the human DMD gene.

TABLE 4
Validation of the mutations in clinical samples. The primers used
for the HRM analysis of the respective patient sample carrying
a known mutation is as listed in the corresponding exon.

| | | | DNA Sequence |
|---|---|---|---|
| | Affected | HRM/Inner | Mutation |
| Patient/Carrier # | Exon | Primers Used | (NM_004006.2) |
| 56 | 37 | Ex37b | c.5510C > T |
| 75 | 7 | Ex7b | c.812C > T |
| 77 (Mother of 75) | 7 | Ex7b | c.812C > T |
| 78 (Brother of 75) | 7 | Ex7b | c.812C > T |
| 100 | 24 | Ex24 | c.3503C > T |
| 128 | 6 | Ex6b | c.677C > T |
| 274 | 46 | Ex46b | c.6992C > G |
| 306 | 60 | Ex60a | c.9188C > T |
| 307 (Mother of | 60 | Ex60a | c.9188C > T |
| 306) | | | |
| 325 | 2 | Ex2 | c.321A > G |
| 360 | 2 | Ex2 | c.287G > C |
| 410 | 57 | Ex57a | c.8660C > T |
| 414/418/432 | 30 | Ex30c with Ex30 | c.4394G > T |
| | | Probe 2 | |
| 634 | 7 | Ex7b | c.860C > TG |
| 669 (Sister of 306) | 60 | Ex60a | c.9188C > T |
| 741 (Sister of 306) | 60 | Ex60a | c.9188C > T |
| 750 | 64 | Ex64 | c.9337C > T |

While the invention has been described with reference to specific methods and embodiments, it will be appreciated that various modifications and changes may be made without departing from the invention.

Each document, reference, patent application or patent cited in this text is expressly incorporated herein in their entirety by reference, which means that it should be read and considered by the reader as part of this text. That the document; reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

1. A method of determining sequence variants within a large gene comprising the steps of:
(a) enriching a nucleic acid sample of the large gene with nested primers designed for the large gene;
(b) using the enriched nucleic acid sample for high resolution melt (HRM); and
(c) detecting differential melt profiles during the transition from double strand to single strand with an increase in temperature wherein sequence point mutations within the gene affects the thermal stability and gives a different melt profile from the normal non-mutated gene sequence.

2. The method of claim 1 further comprising the step of making a Whole-Genome Amplification (WGA) to obtain sufficient amounts of genetic templates for DNA analysis prior to enriching the nucleic acid sample.

3. The method of claim 1 or 2 further comprising the step of spiking the DNA being screened with DNA isolated from a phenotypically normal individual in order to induce synthetic heterozygosity.

4. The method of any one of claims 1 to 3 wherein the large gene is the DMD gene that may potentially contribute to muscle disease.

5. The method of claim 4 wherein the muscle disease comprises muscular dystrophy.

6. The method of claim 5 wherein the muscular dystrophy comprises Duchenne muscular dystrophy.

7. The method of claim 5 wherein the muscular dystrophy comprises Becker muscular dystrophy.

8. The method of claim 4 wherein the DMD gene comprises a nucleic acid homologous to a section of SEQ ID. No. 1.

9. The method of any one of the preceding claims further comprising a cleaning step prior to the HRM step.

10. The method of claim 9 wherein the cleaning step comprises washing with a high magnesium concentration.

11. The method of claim 9 wherein the cleaning step comprises exo-nuclease digestion, dephosphorylation treatment or filtration.

12. A kit comprising at least two nested primers specific to a large gene of interest and reagents' for high resolution melt analysis.

13. The kit of claim 12 further comprising reagents' for whole genome amplification.

14. The kit of claim 12 or 13 further comprising DNA isolated from a phenotypically normal individual.

15. The kit of any one of claims 12 to 14 wherein the at least two nested primers are specific to a DMD gene.

16. The kit of claim 15 wherein the at least two nested primers specific to the DMD gene are specific to a nucleic acid homologous to a section of SEQ ID. No. 1.

17. The kit of claim 15 or 16 wherein the at least two nested primers specific to the DMD gene are selected from any one of the primers listed in table 1 or 3.

18. The kit of any one of claims 13 to 17 wherein the reagents for whole genome amplification comprise an agent for polymerization.

19. The kit of any one of claims 12 to 18 wherein the reagents' for high resolution melt analysis comprise intercalating DNA dyes.

20. The kit of any one of claims 12 to 19 further comprising a washing mix high in magnesium.

21. The kit of any one of claims 12 to 19 further comprising an exo-nuclease.

22. The kit of any one of claims 12 to 21 to rapidly detect mutations that may potentially contribute to muscle disease.


SEQUENCE LISTING (2 sequences)

SEQ ID NO: 1 - DNA, 13993 bp, Homo sapiens
tcctggcatcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaagacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccgagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttac

SEQ ID NO: 2 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM